SEQUENCE LISTING (8 sequences)

SEQ ID NO: 1 - protein, 66 aa, Homo sapiens
GLAGGSAQSQRAPDRVICHSGQQQGLPRAAGGSVPHPRCHCGAGRADWPGPPELDVCVEEAEGEAP

SEQ ID NO: 2 - protein, 14 aa, Homo sapiens
ELDVCVEEAEGEAP

SEQ ID NO: 3 - protein, 18 aa, Homo sapiens
ELQLEESCAEAQDGELDG

SEQ ID NO: 4 - protein, 32 aa, Homo sapiens
GSCSVADWQMPPPYVVLDLPQETLEEETPGAN

SEQ ID NO: 5 - protein, 32 aa, Homo sapiens
GSCCVADWQMPPPYVVLDLPQETLEEETPGAN

SEQ ID NO: 6 - protein, 26 aa, Homo sapiens
DWQMPPPYVVLDLPQETLEEETPGAN

SEQ ID NO: 7 - protein, 13 aa, Homo sapiens
EGEVSADEEGFEN

SEQ ID NO: 8 - protein, 27 aa, Homo sapiens
YLAMTPLIPQSKDENSDDYTTFDDVGS

(19) United States
(12) Patent Application Publication
(10) Pub. No.: US 2009/0220416 A1
(43) Pub. Date: Sep. 3, 2009

(54) IMMUNOGLOBULIN ASSOCIATED CELL-SURFACE DETERMINANTS IN THE TREATMENT OF B-CELL DISORDERS

(76) Inventors: Sydney Welt, Armonk, NY (US); David A. Kostyal, Sayre, PA (US)

Correspondence Address: GOTTLIEB RACKMAN & REISMAN PC, 270 MADISON AVENUE, 8TH FLOOR, NEW YORK, NY, 10016-0601, US

(21) Appl. No.: 12/298,781

(22) PCT Filed: May 3, 2007
(86) PCT No.: PCT/US07/68174

§ 371 Date: Oct. 28, 2008

Related U.S. Application Data
(60) Provisional application No. 60/797,286, filed on May 3, 2006.

(51) Int. Cl. A61K 51/10; A61K 39/395; C12Q 1/68; C12Q 1/00

(52) U.S. Cl. 424/1.49; 424/131.1; 435/6; 435/4

(57) ABSTRACT

The present invention provides methods, compositions and vaccines for specifically targeting immunoglobulin associated cell-surface determinants that are not shed into the blood of a host. In some cases, the immunoglobulin associated cell-surface determinants will be involved in various B-cell disorders. The methods involve administering an IACSD targeting element to a B-cell. The method can employ treating an individual with a B-cell associated disorder by administering an effective amount of an IACSD targeting preparation. The compositions include isolated antibodies where the antibodies associate with an immunoglobulin associated cell-surface determinants. The invention also includes vaccines that immunize against immunoglobulin associated cell-surface determinants.

### FIELD OF THE INVENTION

The present invention relates to compositions and methods for targeting immunoglobulin associated cell-surface determinants (IACSDs) restricted to all subsets of B-cell lineage cells, including the IACSDs encoded by SEQ ID NOS.: 1-8, and their use in the therapy and diagnosis of various natural and pathological states associated with the subset of B-cells, including cancer, autoimmune disease, organ transplant rejection, and allergic reactions.

### BACKGROUND OF THE INVENTION

Targeted drugs that selectively bind to defined cell determinants have been successfully developed as diagnostic and therapeutic agents. In oncology and autoimmune diseases, these drugs have proven their effectiveness in various tumor types and immune disorders and several targeted drugs have been approved for use in a clinical setting. Often, this new class of medicinals has the advantage of lower toxicity, as fewer non-specific interactions as compared to traditional medicinals is encountered. Rituxan is a prime example of this new class of drugs. A subset of these targeted drugs bind to cell surface determinants, avoiding resistant mechanisms related to cell membrane transport and allowing for recruitment of immune effector mechanisms as part of the therapeutic modality or to act as vehicles to direct and deliver toxic moieties to tumor or targeted normal tissues.

The feasibility of targeted drug approaches to treat cancer, immune or other diseases have depended largely on the relative uniqueness of the defined target determinants. Generally, the conditions necessary for success include efficient targeting of the cells responsible for the disease etiology, the ability specifically to deliver a toxic moiety to the targeted cells, blocking of the critical functional aspect of the targeted molecule and in some cases, the activation of an immune response directed to the targeted cell. It has been generally accepted that the targeted determinant would also need to be absolutely specific to the tumor or pathologic tissue. Unfortunately, only a limited number of cell determinants have been discovered that are truly tumor or pathologic tissue specific. Therefore targeted drugs based on absolute “tumor or pathologic tissue” specificities may be so rare as to limit the number of disease entities that are treatable. Conversely lowering the level of targeting specificity reduces the effectiveness of new drugs by virtue of their non-specific normal tissue reactivity inducing toxicity. Thus drugs with “relative” specificity may prove the best balance between effectiveness and toxicity.

Due to the lack of feasibility of treating a significant number of pathologies by specifically targeting determinants on tumors or pathological tissue, a new method for designing targeted drugs is needed. In the present invention, as an alternative to specifically targeting tumor or pathological tissue determinants, we set forth methods to design targeted drugs to determinants on the cell surface of all specific B-cell lineages, and methods to treat pathologies based on these targeted drugs.

### SUMMARY OF THE INVENTION

The present invention provides methods and compositions that make use of a family of immunoglobulin associated cell-surface determinants (IACSDs) having absolute specificity for subsets of the “B-cell lineage” of immune cells for the treatment of a variety of diseases. More specifically, this invention relies on defined proteins and peptides found on the surface of B-cell or neoplastic B-cells as well as to nucleic acid molecules encoding the sequence of said proteins and peptides, as targeting sites for various targeting elements. The proteins and peptides can be detected and targeted with targeting elements that include, but are not limited to, specific antibodies, antibody-based constructs, peptides and/or drugs specifically selected for binding to these said proteins or peptides. In some instances, the surface peptides have molecular weights of approximately 150,000-200,000 based on values predicted from the said sequences from the nucleic acid code.

The targeting elements and compositions containing the targeting element may be used to treat an individual having B-cells such as a mammal (e.g., a human or other individual such as primate, rodent, pig, dog or cat) by administering an effective amount of the targeting element to the host.

This invention also relates to the use of these said nucleic acid molecules or proteins in monomeric or multimeric forms and to antibodies, antibody-based constructs, specific binding peptides or specific binding drugs in diagnostic or screening in vitro or in vivo and in therapeutic methods.

The specific IASCDs of the present invention are not shed into the blood of a host which is important characteristic of determinants for successful drug targeting. Circulating target molecules in the blood can bind drug and divert drug to pathways for metabolism or excretion such as the liver or the kidney. Secreted target determinants can also result in drug initially targeted to tumor being re-released into the blood as the determinant is shed from the cell surface.

The IASCDs that form the basis of this invention are expressed in all B-cell derived neoplasms including the most differentiated form, plasma cell neoplasms such as lymphoma and leukemia and plasma cell dyscrasias such as multiple myeloma. These determinants are more specific than currently approved agents for the targeting of B-cell neoplasms, as the IASCDs sub-divides B-cell differentiation allowing for the specific targeting for modulation or killing of subsets of normal or malignant B-cells respectively. Currently approved drugs for the treatment of lymphomas react with a large proportion of the B-cell compartments (e.g., CD20 expressing cells) resulting in reactions with and/or lysis of large amounts of normal cells. The expression of IASCDs by the complete B-cell lineage allows for the wider use of these agents in B-cell malignancies or B-cell dependent immune suppression for autoimmune disease. However, as a consequence, these IASCDs which subdivide the B cell lineage are each a targeted therapeutic reagent, which will have reactivity with a small subset of normal cells. Reagents with more defined specificity are needed to reduce normal tissue lysis.

Biological effects induced by binding to IACSDs include, but are not limited to, growth inhibition, cell cycle perturbation, growth stimulation, differentiation, senescence, morphological changes, apoptosis, anti-migration, anti-angiogenesis, induction of new protein synthesis, complex internalization, endocytosis, protein metabolism, growth factor blockade, increased drug sensitivity, protein synthesis inhibition, reversal of immune suppression, specific immune suppression, antigen presentation, T-cell stimulation, cytokine secretion, induction of inflammation, activation of coagulation, thrombosis and consumption of clotting factors and prostaglandin biosynthesis.

While the IASCDs defined here offer a more defined and restricted target for development of new diagnostic or therapeutic reagents, their use in combination with currently available therapeutics also makes it possible to produce additive or synergistic combinations allowing for the successful treatment of subsets of B-cell neoplasms that are not yet treatable.

The IASCDs defined here under certain circumstances may be induced to internalize into the B cell, when antibodies or other binding moieties bind to the cell surface determinants. This process will allow for the specific delivery of cofactors attached to the targeting agent to the cell surface and subsequently the internalization of these complexes will result in cofactor entry into the cell. These cofactors include other proteins, nucleic acids, carbohydrates, drugs or radioactive substances, for use as therapeutic, diagnostic, imaging or screening reagents.

The development of immunoglobulins detecting and binding to the IASCDs will result in immune-mediated destruction of targeted B cells. This may occur by complement or cell mediated effects as has been described for other antibody constructs.

The lack of homology of said nucleic acid and amino acid sequences suggests that significant specificity and low cross reactivity with other proteins will allow low toxicity for developed reagents.

Some methods of the present invention involve administering an agent to an individual. The methods comprise administering a targeting preparation comprising a targeting element to the individual, wherein the targeting element targets an immunoglobulin associated cell surface determinant on a B cell that is not shed into the blood of the host or present in the corresponding secreted Ig. The B-cell may be a neoplastic B cell. In some methods, the targeting element may comprise a targeting antibody or antibody fragment, wherein the targeting antibody or antibody fragment specifically recognizes an immunoglobulin associated cell-surface determinant on a B cell that is not shed into the blood of the individual or present in the corresponding secreted immunoglobulin. The targeting antibody or antibody fragment may be humanized. Typically, the targeting element specifically recognizes an immunoglobulin associated cell-surface determinant on a B cell that is not shed into the blood of the individual or present in the corresponding secreted Ig. The methods may involve administering the targeting element to a B-cell in vivo.

In some methods, the immunoglobulin associated cell-surface determinant is a peptide associated with an immunoglobulin isotype, including any of IgA, IgD, IgE, IgG, and IgM. For example, the IASCD may be a peptide comprising any one of SEQ ID NOS: 1-8. The immunoglobulin associated cell-surface determinant may also be immunogenic fragments of such peptides or variants thereof having at least 80%, at least 85%, at least 90%, or at least 95% amino acid identity to the peptide.

In some methods, the individual may be administered a therapeutically effective amount of a cytotoxic agent with the targeting preparation, wherein the cytotoxic agent and the targeting element may be administered in any order or concurrently. The cytotoxic agent and the targeting element may form a conjugate.

In some methods, at least one additional targeting agent may be administered, wherein the at least one additional targeting agent targets a determinant on a B-cell. The determinant on a B cell may comprise the CD20 epitope.

Compositions of the present invention include a targeting composition. A targeting composition may comprise an isolated targeting antibody, antigen binding fragment, or antibody fragment, wherein the isolated antibody or antigen binding fragment associates with an immunoglobulin associated cell surface determinant on a B cell that is not shed into the blood of a host or present in the corresponding secreted Ig. The composition may further comprise a cytotoxic agent, which may be a chemotherapeutic agent or a radionuclide. The antibody, antigen-binding fragment, or antibody fragment may be conjugated to the cytotoxic agent. The antibody, antigen binding fragment, or antibody fragment may inhibit one or more functions associated with the immunoglobulin associated cell surface determinant.

In some compositions, at least one additional targeting agent may be administered, wherein the at least one additional targeting agent targets a determinant on a B-cell. The determinant on a B cell may comprise the CD20 epitope.

Some methods may involve diagnosing a B cell disorder. A method of diagnosing a B-cell disorder may comprise obtaining a sample from an individual having or suspected of having a B cell disorder, detecting or measuring in the sample the expression of an immunoglobulin associated cell surface determinant protein, that is not shed into the blood of a host or present in the corresponding secreted Ig, on a cell or the expression of an immunoglobulin associated cell surface determinant nucleic acid in a cell and comparing the expression to a standard. The expression of the immunoglobulin associated cell surface determinant protein or nucleic acid relative to the standard may be correlated to a B cell disorder.

Some embodiments of the present invention include a vaccine. The vaccine for treating B-cell disorders may comprise a targeting preparation comprising a targeting element that targets an immunoglobulin associated cell surface determinant, that is not shed into the blood of a host or present in the corresponding secreted Ig, and a physiologically acceptable carrier. The vaccine may further comprise physiologically acceptable carrier comprises an adjuvant or an immunostimulatory agent.

### BRIEF DESCRIPTION OF THE DRAWING

FIG. 1 presents data showing the interaction of an anti-IACSD antibody with a B-cell in accordance with the present invention. Panel A is a view of the cells illuminating the fluorescent dye attached to the antibody. Panel B is a whitefield view of the same cells.

### DETAILED DESCRIPTION

The present invention relates to methods of targeting a specific subset of B-cells that express immunoglobulin associated cell-surface determinants (IACSDs) using targeting elements, such as IACSD-binding polypeptides, nucleic acids encoding IACSD, anti-IACSD antibodies, including fragments or engineered products or other modifications of any of these elements. In some embodiments, these targeting elements will be administered to B-cells that are either in vivo or in vitro. The IACSDs of interest for the present invention are extracellular determinants that are not present on immunoglobulins circulating in the blood of host. Therefore, targeting elements that specifically target these IACSDs are capable of binding to the antibodies on tumor cells without binding to circulating immunoglobulin molecules.

Although as a category, IACSDs generally cover any immunoglobulin associated peptide specifically expressed by a particular subset of B-cells, but not found on the corresponding secreted Ig. IACSD peptides may be associated with any and all types of immunoglobulin isotype. For example, the peptides in SEQ ID NOS: 1-8 encompass peptides associated with IgE, IgG, IgA, IgM, and IgD. Non-limiting examples of specific sequences for IACSDs useful for targeting in accordance with the present invention, include the following peptides:

TABLE 1
Exemplary IACSD Peptides

| Immuno- | | |
|---|---|---|
| globulin | SEQ ID NO | Peptide Sequence |
| IgE | SEQ ID NO: 1 | GLAGGSAQSQRAPDRVICHSGQQQGLPRA |
| | | AGGSVPHPRCHCGAGRADWPGPPELDVCV |
| | | EEAEGEAP |
| IgE | SEQ ID NO: 2 | ELDVCVEEAEGEAP |
| IgG | SEQ ID NO: 3 | ELQLEESCAEAQDGELDG |
| IgA | SEQ ID NO: 4 | GSCSVADWQMPPPYVVLDLPQETLEEETP |
| | | GAN |
| IgA | SEQ ID NO: 5 | GSCCVADWQMPPPYVVLDLPQETLEEETP |
| | | GAN |
| IgA | SEQ ID NO: 6 | DWQMPPPYVVLDLPQETLEEETPGAN |
| IgM | SEQ ID NO: 7 | EGEVSADEEGFEN |
| IgD | SEQ ID NO: 8 | YLAMTPLIPQSKDENSDDYTTFDDVGS |

In certain embodiments, the present invention provides a novel approach for diagnosing and treating diseases and disorders associated with IACSD-expressing B-cells. This approach comprises administering to an individual an effective amount of targeting preparations such as vaccines, antigen presenting cells, or pharmaceutical compositions comprising the targeting elements, such as engineered constructs, IACSD-binding polypeptides, nucleic acids encoding SEQ ID NOS 1-8, and/or anti-IACSD antibodies. In many cases, targeting of IACSD on the cell membranes of IACSD-expressing B-cells may inhibit the growth of or destroy such cells. Generally, an effective amount to inhibit the growth of or destroy the IACSD-expressing B-cells will be the amount of such IACSD targeting preparations necessary to target the IACSD on the cell membrane and inhibit the growth of or destroy the IACSD-expressing B-cells.

A further embodiment of the present invention enhances the effects of therapeutic agents and adjunctive agents used to treat and manage disorders associated with IACSD-expressing B-cells, by administering IACSD preparations with therapeutic and adjuvant agents commonly used to treat B-cell disorders. For example, chemotherapeutic agents useful in treating neoplastic disease and antiproliferative agents and drugs used for immunosuppression may include alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as triethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophyllotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone, and anthracycline, and proteasome inhibitors and thalidomide or anti-angiogenic agents.

Further, adjunctive therapy used in the management of B-cell disorders includes, for example, radiosensitizing agents, coupling of antigen with heterologous proteins, such as globulin or beta-galactosidase, or inclusion of an adjuvant during immunization.

In some cases, high doses may be required for some therapeutic agents to achieve levels to effectuate the target response. However, these high doses may also be associated with a greater frequency of dose-related adverse effects. In contrast, combined use of the methods of the present invention that specifically target B-cells expressing IACSD with agents commonly used to treat B-cell related disorders allows the use of relatively lower doses of such agents, which may result in a lower frequency of adverse side effects commonly associated with long-term administration of the conventional therapeutic agents. Thus, another indication for the methods of this invention is to reduce adverse side effects associated with conventional therapy of disorders associated with IACSD-expressing B-cells.

### DEFINITIONS

As used herein the term “antibody” refers to an immunoglobulin and any antigen-binding portion of an immunoglobulin (e.g. IgG, IgD, IgA, IgM and IgE) i.e., a polypeptide that contains an antigen binding site, which specifically binds (“immunoreacts with”) an antigen. Antibodies can comprise at least one heavy (H) chain and at least one light (L) chain inter-connected by at least one disulfide bond. The term “V_{H}” refers to a heavy chain variable region of an antibody. The term “V_{L}” refers to a light chain variable region of an antibody. In exemplary embodiments, the term “antibody” specifically covers monoclonal and polyclonal antibodies. A “polyclonal antibody” refers to an antibody which has been derived from the sera of animals immunized with an antigen or antigens. A “monoclonal antibody” refers to an antibody produced by a single clone of hybridoma cells. Techniques for generating monoclonal antibodies include, but are not limited to, the hybridoma technique (see Kohler & Milstein (1975) Nature 256:495 497); the trioma technique; the human B-cell hybridoma technique (see Kozbor, et al. (1983) Immunol. Today 4:72), the EBV hybridoma technique (see Cole, et al., 1985 In: Monoclonal Antibodies and Cancer Therapy, Alan R. Liss, Inc., pp. 77 96) and phage display.

The term “fragment” of a nucleic acid refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 100 nucleotides, preferably less than about 75 nucleotides, more preferably less than about 100 nucleotides, more preferably less than about 50 nucleotides and most preferably less than 30 nucleotides. In certain embodiments, the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. In some embodiments, the fragment comprises a sequence substantially similar to a portion of IACSD. Generally, substantially similar includes sequences that share at least 85%, and preferably greater than 95% sequence similarity.

A polypeptide “fragment” is a stretch of amino acid residues of at least about 5 amino acids, preferably at least about 7 amino acids, more preferably at least about 9 amino acids and most preferably at least about 13 or more amino acids. The peptide preferably is less than about 35 amino acids, more preferably less than 32 amino acids. In many embodiments, the peptide is from about five to about 35 amino acids. To be active, any polypeptide must have sufficient length to display biological and/or immunological activity. The term “immunogenic” refers to the capability of the natural, recombinant or synthetic IACSD-like peptide, or any peptide thereof, to induce a specific immune response in appropriate animals or cells and to bind with specific antibodies.

The term “variant” (or “analog”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence. In some embodiments, the polypeptide or polypeptide fragment comprises variants having at least 80%, at least 85%, at least 90%, or at least 95% amino acid identity to a naturally occurring polypeptide. Percentage identity is calculated by determining the number of positions at which the identical amino acid residue occurs in both sequences to yield the number of matched positions. Algorithms for aligning sequences and calculating percentage identity are well-known in the art (e.g. BLAST).

Alternatively, recombinant variants encoding these same or similar polypeptides may be synthesized or selected by making use of the “redundancy” in the genetic code. Various codon substitutions, such as the silent changes that produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

### Immunotargeting of IACSDs

Immunotargeting may also involve the administration of engineered products, binding peptides, or components of the immune system, such as antibodies, antibody fragments, or primed cells of the immune system against the target. Anti-CD20 and anti-CD22 antibodies are two examples of suitable antibodies. Methods of immunotargeting cancer cells using antibodies or antibody fragments are well known in the art. For example, U.S. Pat. No. 6,306,393 describes the use of anti-CD22 antibodies in the immunotherapy of B-cell malignancies, and U.S. Pat. No. 6,329,503 describes immunotargeting of cells that express serpentine transmembrane antigens.

IACSD antibodies (including humanized or human monoclonal antibodies or fragments or other modifications thereof, optionally conjugated to cytotoxic agents) may be introduced into a individual such that the antibody binds to IACSD expressed by B-cells and mediates the destruction of the cells and/or inhibits the growth of the cells. Without intending to limit the disclosure, mechanisms by which such antibodies can exert a therapeutic effect may include complement-mediated cytolysis, antibody-dependent cellular cytotoxicity (ADCC), modulating the physiologic function of IACSDs, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis. IACSD antibodies conjugated to toxic or therapeutic agents, such as radioligands or cytosolic toxins, may also be used therapeutically to deliver the toxic or therapeutic agent directly to IACSD-bearing B-cells.

In certain embodiments, IACSD antibodies may be used to suppress the immune system in patients receiving organ transplants or in patients with autoimmune diseases such as arthritis. Healthy immune cells would be targeted by these antibodies leading to their death and clearance from the system, thus suppressing the immune system by specifically blocking production of IgG, IgM, IgA or IgE.

Although anti-IACSD antibody therapy may be useful for all stages of cancers of a subset of B-cell lineage, antibody therapy may be particularly appropriate in advanced or metastatic cancers. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with tumors resistant to chemotherapeutic agents with anti-IACSD antibody might induce sensitivity and responsiveness to these agents in combination.

Prior to anti-IACSD immunotargeting, a patient may be evaluated for the presence and level of IACSD expression by the tumor cells, preferably using immunohistochemical assessments of tumor tissue, quantitative IACSD imaging, quantitative RT-PCR, or other techniques capable of reliably indicating the presence and degree of IACSD expression. For example, a blood or biopsy sample may be evaluated by immunohistochemical methods to determine the presence of IACSD-expressing B-cells or to determine the extent of IACSD expression on the surface of the cells within the sample. Methods for immunohistochemical analysis of tumor tissues are generally well known in the art.

Anti-IACSD antibodies useful in treating cancers include those that are capable of initiating a potent immune response against the tumor and those, which are capable of direct cytotoxicity. In this regard, anti-IACSD mAbs may elicit tumor cell lysis by either complement-mediated or ADCC mechanisms, both of which require an intact Fc portion of the immunoglobulin molecule for interaction with effector cell Fc receptor sites or complement proteins. In addition, anti-IACSD antibodies that exert a direct biological effect on tumor growth are useful in the practice of the invention. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-IACSD antibody exerts an anti-tumor effect may be evaluated using any number of in vitro assays designed to determine ADCC, ADMMC, complement-mediated cell lysis, and so forth, as is generally known in the art.

The anti-tumor activity of a particular anti-IACSD antibody, or combination of anti-IACSD antibody, may be evaluated in vivo using a suitable animal model. For example, xenogenic lymphoma cancer models where human lymphoma cells are introduced into immune compromised animals, such as nude or SCID mice may be used for evaluation. Efficacy may be predicted using assays, which measure inhibition of tumor formation, tumor regression or metastasis, and the like.

It should be noted that the use of murine or other non-human monoclonal antibodies, human/mouse chimeric mAbs are generally disfavored because they are ineffective at delivering antibodies to the tumors. In addition, these non-human monoclonal antibodies may induce moderate to strong immune responses in some patients. In the most severe cases, such an immune response may lead to the extensive formation of immune complexes, which, potentially, can cause tissue damage, such as renal failure. Accordingly, preferred monoclonal antibodies used in the practice of the therapeutic methods of the invention are those which are either fully human or humanized and which bind specifically to the target IACSD antigen with high affinity but exhibit low or no antigenicity in the patient.

The method of the invention contemplates the administration of single anti-IACSD monoclonal antibodies (mAbs) as well as combinations, or “cocktails,” of different mAbs. Two or more monoclonal antibodies that bind to IACSD may provide an improved effect compared to a single antibody. Alternatively, a combination of an anti-IACSD antibody with an antibody that binds a different antigen may provide an improved effect compared to a single antibody. Such mAb cocktails may have certain advantages inasmuch as they contain mAbs, which exploit different effector mechanisms or combine directly cytotoxic mAbs with mAbs that rely on immune effector functionality. Such mAbs in combination may exhibit synergistic therapeutic effects. In addition, the administration of anti-IACSD mAbs may be combined with other therapeutic agents, including but not limited to various chemotherapeutic agents, androgen-blockers, and immune modulators (e.g., IL-2, GM-CSF). The anti-IACSD mAbs may be administered in their “naked” or unconjugated form, or may have therapeutic agents conjugated to them. Additionally, bispecific antibodies may be used. Such an antibody would have one antigenic binding domain specific for IACSD and the other antigenic binding domain specific for another antigen (such as CD20 for example). Finally, Fab IACSD antibodies or fragments of these antibodies (including fragments conjugated to other protein sequences or toxins) may also be used as therapeutic agents.

### 1. Anti-IACSD Antibodies

Antibodies that specifically bind IACSDs are useful in compositions and methods for immunotargeting a subset of B-cells expressing IACSDs and for diagnosing a disease or disorder wherein a subset of B-cells involved in the disorder express IACSDs. An example of a subset of B-cells that express IACSDs includes plasma cells and cells of plasma cell lineage. Such antibodies include monoclonal and polyclonal antibodies, single chain antibodies, chimeric antibodies, bifunctional/bispecific antibodies, humanized antibodies, human antibodies, and complementary determining region (CDR)-grafted antibodies, including compounds that include CDR and/or antigen-binding sequences, which specifically recognize IACSDs. Antibody fragments, including Fab, Fab′, F(ab′)₂, and Fᵥ, and engineered constructs are also useful.

With respect to antibodies and antibody fragments, the term “specific for” or “specifically recognizes” indicates that the variable regions of the antibodies recognize and bind IACSDs (i.e., the variable regions are able to distinguish IACSDs from other similar polypeptides despite sequence identity, homology, or similarity found in the family of polypeptides). An antibody “specifically recognizes” an antigen or an epitope of an antigen if the antibody binds preferably to the antigen over most other antigens. Typically specific binding results in a much stronger association between the antibody binding site and the target antigen than between the antibody binding site and non-target molecule. For specific binding, the affinity constant of the antibody binding site for its cognate antigen may be at least 10⁷, at least 10⁸, at least 10⁹, preferably at least 10¹⁰, or more preferably at least 10¹¹ liters/mole. Screening assays in which one can determine binding specificity of an anti-IACSD antibody are well known and routinely practiced in the art. For an example of how to determine the binding specificity of an antibody, see Chapter 6, Antibodies A Laboratory Manual, Eds. Harlow, et al., Cold Spring Harbor Laboratory; Cold Spring Harbor, N.Y. (1988)).

IACSD-binding polypeptides can be used to immunize animals to obtain polyclonal and monoclonal antibodies that specifically react with IACSDs. Such antibodies can be obtained using either the entire protein or fragments thereof as an immunogen. The peptide immunogens additionally may contain a cysteine residue at the carboxyl terminus and the peptide immunogens may be conjugated to a hapten such as keyhole limpet hemocyanin (KLH). Methods for synthesizing such peptides have been previously described (Merrifield, J. Amer. Chem. Soc. 85, 2149-2154 (1963); Krstenansky, et al., FEBS Lett. 211: 10 (1987)). Techniques for preparing polyclonal and monoclonal antibodies as well as hybridomas capable of producing the desired antibody have also been previously disclosed (Campbell, Monoclonal Antibodies Technology: Laboratory Techniques in Biochemistry and Molecular Biology, Elsevier Science Publishers, Amsterdam, The Netherlands (1984); St. Groth et al., J. Immunol. 35:1-21 (1990); Kohler and Milstein, Nature 256:495-497 (1975)), the trioma technique, the human B-cell hybridoma technique (Kozbor et al., Immunology Today 4:72 (1983); Cole et al., in, Monoclonal Antibodies and Cancer Therapy, Alan R. Liss, Inc., pp. 77-96 (1985)).

Any animal capable of producing antibodies can be immunized with an IACSD peptide or polypeptide. Methods for immunization include subcutaneous or intraperitoneal injection of the polypeptide. The amount of the IACSD peptide or polypeptide used for immunization depends on the animal that is immunized, antigenicity of the peptide and the site of injection. The IACSD peptide or polypeptide used as an immunogen may be modified or administered in an adjuvant in order to increase the protein's antigenicity. Methods of increasing the antigenicity of a protein are well known in the art and include, but are not limited to, coupling the antigen with a heterologous protein (such as globulin or galactosidase) or through the inclusion of an adjuvant during immunization.

In some embodiments, antibodies will be generated to IACSDs using a phage display methods known in the art. Examples of references demonstrating generating antibodies using phage display include Huie et al., Proc. Natl. Acad. USA 98(5): 2682-2687 (2001) and Liu et al., J. Mol. Biol. 315: 1063-1073 (2002). An advantage to using phage technology over traditional antibody production via an animal model is that some IACSD peptides may not be immunogenic in a particular animal and phage display technology allows specific insight into peptide-peptide binding interactions that can then be engineered into “human” antibodies.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell that produces an antibody with the desired characteristics. These include screening the hybridomas with an ELISA assay, Western blot analysis, or radioimmunoassay (Lutz et al., Exp. Cell Res. 175:109-124 (1988)). Hybridomas secreting the desired antibodies are cloned and the class and subclass are determined using procedures known in the art (Campbell, A. M., Monoclonal Antibody Technology: Laboratory Techniques in Biochemistry and Molecular Biology, Elsevier Science Publishers, Amsterdam, The Netherlands (1984)). Techniques described for the production of single chain antibodies can be adapted to produce single chain antibodies to IACSD. Generally, techniques for single chain antibodies are demonstrated in U.S. Pat. No. 4,946,778.

For polyclonal antibodies, antibody-containing antiserum is isolated from the immunized animal and is screened for the presence of antibodies with the desired specificity using one of the above-described procedures.

Because antibodies from rodents tend to elicit strong immune responses against the antibodies when administered to a human, it may be advantageous to use non-rodent antibodies. Methods of producing antibodies that do not produce a strong immune response against the administered antibodies are well known in the art. For example, the anti-IACSD antibody can be a nonhuman primate antibody. Methods of making such antibodies in baboons are disclosed in WO 91/11465 and Losman et al., Int. J. Cancer 46:310-314 (1990).

In one embodiment, the anti-IACSD antibody is a humanized monoclonal antibody. The term “humanized antibody” (HuAb) refers to a chimeric antibody with a framework region substantially identical (i.e., at least 85%) to a human framework, having CDRs from a non-human antibody, and in which any constant region has at least about 85 90%, and preferably about 95% polypeptide sequence identity to a human immunoglobulin constant region. See, for example, PCT Publication WO 90/07861 and European Patent No. 0451216. All parts of such a HuAb, except possibly the CDRs, are substantially identical to corresponding parts of one or more native human immunoglobulin sequences. Methods of producing humanized antibodies have been previously described. (U.S. Pat. Nos. 5,997,867 and 5,985,279, Jones et al., Nature 321:522 (1986); Riechmann et al., Nature 332:323 (1988); Verhoeyen et al., Science 239:1534-1536 (1988); Carter et al., Proc. Nat'l Acad. Sci. USA 89:4285-4289 (1992); Sandhu, Crit. Rev. Biotech. 12:437-462 (1992); and Singer et al., J. Immun. 150:2844-2857 (1993)). Antibody humanization may be performed by CDR-grafting, which involves the genetic transfer of mouse CDRs (which are responsible for antigen binding) into human frameworks of a variable region. CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. This procedure is described in detail in Larrick et al., Methods: A Companion to Methods in Enzymology 2:106 (1991); Courtenay-Luck, pp. 166-179 in, Monoclonal Antibodies Production, Engineering and Clinical Applications, Eds. Ritter et al., Cambridge University Press (1995); and Ward et al., pp. 137-185 in, Monoclonal Antibodies Principles and Applications, Eds. Birch et al., Wiley-Liss, Inc. (1995). The humanized antibodies that contain the mouse CDRs are produced by transgenic mice that have been engineered to produce human antibodies. Hybridomas derived from such mice will secrete large amounts of humanized monoclonal antibodies. Methods for obtaining humanized antibodies from transgenic mice are described in Green et al., Nature Genet. 7:13-21 (1994), Lonberg et al., Nature 368:856 (1994), and Taylor et al., Int. Immun. 6:579 (1994).

The present invention also includes the use of anti-IACSD antibody fragments. Antibody fragments can be prepared by proteolytic hydrolysis of an antibody or by expression in E. coli of the DNA coding for the fragment. Antibody fragments can be obtained by pepsin or papain digestion of whole antibodies. For example, antibody fragments can be produced by enzymatic cleavage of antibodies with pepsin to provide a fragment denoted F(ab′)₂. This fragment can be further cleaved using a thiol reducing agent, and optionally a blocking group for the sulfhydryl groups resulting from cleavage of disulfide linkages, to produce Fab′ monovalent fragments. Alternatively, an enzymatic cleavage using pepsin produces two monovalent Fab fragments and an Fc fragment directly. These methods have been previously described in U.S. Pat. Nos. 4,036,945 and 4,331,647, Nisonoff et al., Arch Biochem. Biophys. 89:230 (1960); Porter, Biochem. J. 73:119 (1959), and Edelman et al., Meth. Enzymol. 1:422 (1967). Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody. For example, Fv fragments comprise an association of V_{H} and V_{L} chains, which can be noncovalent. Alternatively, the variable chains can be linked by an intermolecular disulfide bond or cross-linked by chemicals such as glutaraldehyde.

In one embodiment, the Fv fragments comprise V_{H} and V_{L} chains that are connected by a peptide linker. These single-chain antigen-binding proteins (sFv) are prepared by constructing a structural gene comprising DNA sequences encoding the V_{H} and V_{L} domains that are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing sFvs have been previously described in U.S. Pat. No. 4,946,778; Whitlow et al., Methods: A Companion to Methods in Enzymology 2:97 (1991); Bird et al., Science 242:423 (1988); and Pack et al., Bio/Technology 11:1271 (1993).

The present invention further provides the above-described antibodies in detectably labeled form. Antibodies can be detectably labeled with radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Procedures for accomplishing such labeling have been previously disclosed in depth in Sternberger et al., J. Histochem. Cytochem. 18:315 (1970); Bayer et al., Meth. Enzym. 62:308 (1979); Engval et al., Immunol. 109:129 (1972); and Goding, J. Immunol. Meth. 13:215 (1976). Labeled antibodies can be used for in vitro, in vivo, and in situ assays to identify B-cells in which IACSD is expressed.

### 2. Anti-IACSD Antibody Conjugates

The present invention contemplates the use of “naked” anti-IACSD antibodies, as well as the use of immunoconjugates. Immunoconjugates can be prepared by indirectly conjugating a therapeutic agent such as a cytotoxic agent to an antibody component. Toxic moieties include, for example, plant toxins, such as abrin, ricin, modeccin, viscumin, pokeweed anti-viral protein, saporin, gelonin, momoridin, trichosanthin, barley toxin; bacterial toxins, such as Diptheria toxin, Pseudomonas endotoxin and exotoxin, Staphylococcal enterotoxin A; fungal toxins, such as α-sarcin, restrictocin; cytotoxic RNases, such as extracellular pancreatic RNases; DNase I; calicheamicin, and radioisotopes, such as ³²P, ⁶⁷Cu, ⁷⁷As, ¹⁰⁵Rh, ¹⁰⁹Pd, ¹¹¹Ag, ¹²¹Sn, ¹³¹I, ¹⁶⁶Ho, ¹⁷⁷Lu, ¹⁸⁶Re, ¹⁸⁸Re, ¹⁹⁴, and ¹⁹⁹Au. As an example, in humans, clinical trials are underway utilizing a yttrium-90 conjugated anti-CD20 antibody for B-cell lymphomas (Cancer Chemother. Pharmacol. 48(Suppl 1):S91-S95 (2001)).

General techniques for conjugation to therapeutic agents have been previously described in U.S. Pat. Nos. 6,306,393 and 5,057,313, Shih et al., Int. J. Cancer 41:832-839 (1988); and Shih et al., Int. J. Cancer 46:1101-1106 (1990). The general method involves reacting an antibody component having an oxidized carbohydrate portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form the final conjugate.

The carrier polymer is preferably an aminodextran or polypeptide of at least 50 amino acid residues, although other substantially equivalent polymer carriers can also be used. Preferably, the final immunoconjugate is soluble in an aqueous solution, such as mammalian serum, for ease of administration and effective targeting for use in therapy. Thus, solubilizing functions on the carrier polymer will enhance the serum solubility of the final immunoconjugate. In particular, an aminodextran will be preferred.

The process for preparing an immunoconjugate with an aminodextran carrier typically begins with a dextran polymer, advantageously a dextran of average molecular weight of about 10,000-100,000. The dextran is reacted with an oxidizing agent to affect a controlled oxidation of a portion of its carbohydrate rings to generate aldehyde groups. The oxidation is conveniently effected with glycolytic chemical reagents such as NaIO₄, according to conventional procedures. The oxidized dextran is then reacted with a polyamine, preferably a diamine, and more preferably, a mono- or polyhydroxy diamine. Suitable amines include ethylene diamine, propylene diamine, or other like polymethylene diamines, diethylene triamine or like polyamines, 1,3-diamino-2-hydroxypropane, or other like hydroxylated diamines or polyamines, and the like. An excess of the amine relative to the aldehyde groups of the dextran is used to ensure substantially complete conversion of the aldehyde functions to Schiff base groups. A reducing agent, such as NaBH₄, NaBH₃ CN or the like, is used to effect reductive stabilization of the resultant Schiff base intermediate. The resultant adduct can be purified by passage through a conventional sizing column or ultrafiltration membrane to remove cross-linked dextrans. Other conventional methods of derivatizing a dextran to introduce amine functions can also be used, e.g., reaction with cyanogen bromide, followed by reaction with a diamine.

The aminodextran is then reacted with a derivative of the particular drug, toxin, chelator, immunomodulator, boron addend, or other therapeutic agent to be loaded, in an activated form, preferably, a carboxyl-activated derivative, prepared by conventional means, e.g., using dicyclohexylcarbodiimide (DCC) or a water soluble variant thereof, to form an intermediate adduct. Alternatively, polypeptide toxins such as pokeweed antiviral protein or ricin A-chain, and the like, can be coupled to aminodextran by glutaraldehyde condensation or by reaction of activated carboxyl groups on the protein with amines on the aminodextran.

Chelators for radiometals or magnetic resonance enhancers are well known in the art. Typical are derivatives of ethylenediaminetetraacetic acid (EDTA) and diethylenetriaminepentaacetic acid (DTPA). These chelators typically have groups on the side chain by which the chelator can be attached to a carrier. Such groups include, e.g., benzylisothiocyanate, by which the DTPA or EDTA can be coupled to the amine group of a carrier. Alternatively, carboxyl groups or amine groups on a chelator can be coupled to a carrier by activation or prior derivatization and then coupling, all by well-known means.

Boron addends, such as carboranes, can be attached to antibody components by conventional methods. For example, carboranes can be prepared with carboxyl functions on pendant side chains, as is well known in the art. Attachment of such carboranes to a carrier, e.g., aminodextran, can be achieved by activation of the carboxyl groups of the carboranes and condensation with amines on the carrier to produce an intermediate conjugate. Such intermediate conjugates are then attached to antibody components to produce therapeutically useful immunoconjugates, as described below.

A polypeptide carrier can be used instead of aminodextran, but the polypeptide carrier should have at least 50 amino acid residues in the chain, preferably 100-5000 amino acid residues. At least some of the amino acids should be lysine residues or glutamate or aspartate residues. The pendant amines of lysine residues and pendant carboxylates of glutamine and aspartate are convenient for attaching a drug, toxin, immunomodulator, chelator, boron addend or other therapeutic agent. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier and immunoconjugate.

Conjugation of the intermediate conjugate with the antibody component is effected by oxidizing the carbohydrate portion of the antibody component and reacting the resulting aldehyde (and ketone) carbonyls with amine groups remaining on the carrier after loading with a drug, toxin, chelator, immunomodulator, boron addend, or other therapeutic agent. Alternatively, an intermediate conjugate can be attached to an oxidized antibody component via amine groups that have been introduced in the intermediate conjugate after loading with the therapeutic agent. Oxidation is conveniently effected either chemically, e.g., with NaIO₄ or other glycolytic reagent, or enzymatically, e.g., with neuraminidase and galactose oxidase. In the case of an aminodextran carrier, not all of the amines of the aminodextran are typically used for loading a therapeutic agent. The remaining amines of aminodextran condense with the oxidized antibody component to form Schiff base adducts, which are then reductively stabilized, normally with a borohydride reducing agent.

Analogous procedures are used to produce other immunoconjugates according to the invention. Loaded polypeptide carriers preferably have free lysine residues remaining for condensation with the oxidized carbohydrate portion of an antibody component. Carboxyls on the polypeptide carrier can be converted to amines, if necessary, by, e.g., activation with DCC and reaction with an excess of a di amine.

The final immunoconjugate may be purified using conventional techniques, such as sizing chromatography on Sephacryl S-300 or affinity chromatography using one or more IACSD epitopes.

Alternatively, immunoconjugates can be prepared by directly conjugating an antibody component with a therapeutic agent. The general procedure is analogous to the indirect method of conjugation except that a therapeutic agent is directly attached to an oxidized antibody component. It will be appreciated that other therapeutic agents can be substituted for the chelators described herein. Those of skill in the art will be able to devise conjugation schemes without undue experimentation.

As an illustration of a conjugation scheme, a therapeutic agent can be attached at the hinge region of a reduced antibody component via disulfide bond formation. For example, the tetanus toxoid peptides can be constructed with a single cysteine residue that is used to attach the peptide to an antibody component. Alternatively, such peptides can be attached to the antibody component using a heterobifunctional cross-linker, such as N-succinyl 3-(2-pyridyldithio) proprionate (SPDP) as demonstrated in Yu et al., Int. J. Cancer 56:244 (1994). Other references that demonstrate general techniques for such conjugation have been previously described in Wong, Chemistry of Protein Conjugation and Cross-linking, CRC Press (1991); Upeslacis et al., pp. 187-230 in, Monoclonal Antibodies Principles and Applications, Eds. Birch et al., Wiley-Liss, Inc. (1995); and Price, pp. 60-84 in, Monoclonal Antibodies: Production, Engineering and Clinical Applications Eds. Ritter et al., Cambridge University Press (1995).

As described above, carbohydrate moieties in the Fc region of an antibody can be used to conjugate a therapeutic agent. However, the Fc region may be absent if an antibody fragment is used as the antibody component of the immunoconjugate. Nevertheless, it is possible to introduce a carbohydrate moiety into the light chain variable region of an antibody or antibody fragment. Then, the engineered carbohydrate moiety is used to attach a therapeutic agent.

Numerous possible variations of the conjugation methods are known in the art. For example, the carbohydrate moiety can be used to attach polyethyleneglycol in order to extend the half-life of an intact antibody, or antigen-binding fragment thereof, in blood, lymph, or other extracellular fluids. Moreover, it is possible to construct a “divalent immunoconjugate” by attaching therapeutic agents to a carbohydrate moiety and to a free sulfhydryl group. Such a free sulfhydryl group may be located in the hinge region of the antibody component.

### 3. Anti-IACSD Antibody Fusion Proteins

When the therapeutic agent to be conjugated to the antibody is a protein, the present invention contemplates the use of fusion proteins comprising one or more anti-IACSD antibody moieties and an immunomodulator or toxin moiety. Methods of making antibody fusion proteins have been previously described in U.S. Pat. No. 6,306,393. For example, antibody fusion proteins comprising an interleukin-2 moiety have been previously disclosed in Boleti et al., Ann. Oncol. 6:945 (1995), Nicolet et al., Cancer Gene Ther. 2:161 (1995), Becker et al., Proc. Natl. Acad. Sci. USA 93:7826 (1996), Hank et al., Clin. Cancer Res. 2:1951 (1996) and Hu et al., Cancer Res. 56:4998 (1996)). In addition, Yang et al., Hum. Antibodies Hybridomas 6:129 (1995), describe a fusion protein that includes an F(ab′)₂ fragment and a tumor necrosis factor alpha moiety.

Methods of making antibody-toxin fusion proteins in which a recombinant molecule comprises one or more antibody components and a toxin or chemotherapeutic agent also are known in the art. For example, antibody-Pseudomonas exotoxin A fusion proteins have been described in Chaudhary et al., Nature 339:394 (1989); Brinkmann et al., Proc. Nat'l Acad. Sci. USA 88:8616 (1991); Batra et al., Proc. Natl. Acad. Sci. USA 89:5867 (1992); Friedman et al., J. Immunol. 150:3054 (1993); Wels et al., Int. J. Can. 60:137 (1995); Fominaya et al., J. Biol. Chem. 271:10560 (1996); Kuan et al., Biochemistry 35:2872 (1996); and Schmidt et al., Int. J. Can. 65:538 (1996). Similarly, antibody-toxin fusion proteins containing a diphtheria toxin moiety have been described in Kreitman et al., Leukemia 7:553 (1993); Nicholls et al., J. Biol. Chem. 268:5302 (1993); Thompson et al., J. Biol. Chem. 270:28037 (1995); and Vallera et al., Blood 88:2342 (1996). Deonarain et al. (Tumor Targeting 1:177 (1995)), have described an antibody-toxin fusion protein having an RNase moiety, while Linardou et al. (Cell Biophys. 24-25:243 (1994)), produced an antibody-toxin fusion protein comprising a DNase I component. In the art, Gelonin and Staphylococcal enterotoxin-A have been used as the toxin moieties in antibody-toxin fusion proteins (Wang et al., Abstracts of the 209th ACS National Meeting, Anaheim, Calif., Apr. 2-6, 1995, Part 1, BIOT005; Dohlsten et al., Proc. Natl. Acad. Sci. USA 91:8945 (1994)).

A. Targeting Using IACSD Vaccines

One embodiment the present invention provides a vaccine comprising an IACSD-binding polypeptide to stimulate the immune system against IACSDs, thus targeting IACSD-expressing B-cells. Use of a vaccine that specifically targets Immunoglobulin associated cell-surface determinants will be similar to the well-known use of a tumor antigen in a vaccine for generating cellular and humoral immunity for the purpose of anti-cancer therapy. For example, one type of tumor-specific vaccine uses purified idiotype protein isolated from tumor cells, coupled to keyhole limpet hemocyanin (KLH) and mixed with adjuvant for injection into patients with low-grade follicular lymphoma (Hsu, et al., Blood 89: 3129-3135 (1997)). In a similar manner, purified IACSD protein isolated from B-cells could be used in vaccine formulations. Another example of tumor-specific vaccines known in the art includes those depicted in U.S. Pat. No. 6,312,718, which describes methods for inducing immune responses against malignant B-cells, in particular lymphoma, chronic lymphocytic leukemia, and multiple myeloma. The methods described in U.S. Pat. No. 6,312,718 utilize vaccines that include liposomes having (1) at least one B-cell malignancy-associated antigen, (2) IL-2 alone, or in combination with at least one other cytokine or chemokine, and (3) at least one lipid molecule. Similar methods may be used to vaccinate against IACSDs. Typically, methods of vaccinating against IACSDs employ an IACSD-binding polypeptide, which may be a fragment, analog and/or variants.

As another example, dendritic cells, one type of antigen-presenting cell, can be used in a cellular vaccine in which the dendritic cells are isolated from the patient, co-cultured with IACSD antigen and then reinfused as a cellular vaccine (Hsu, et al., Nat. Med. 2:52-58 (1996)).

B. Targeting Using Nucleic Acids Encoding IACSDs

1. Direct Delivery of Nucleic Acids

In some embodiments, a nucleic acid encoding IACSD, or encoding a fragment, analog or variant thereof, within a recombinant vector is utilized. The use of nucleic acids to generate immune responses is known in the art. For instance, immune responses can be induced by injection of naked DNA. For example, plasmid DNA that expresses bicistronic mRNA encoding both the light and heavy chains of tumor idiotype proteins, such as those from B-cell lymphoma, when injected into mice, are able to generate a protective, anti-tumor response (Singh et al., Vaccine 20:1400-1411 (2002)). IACSD viral vectors are particularly useful for delivering IACSD-encoding nucleic acids to cells. Examples of vectors include those derived from influenza, adenovirus, vaccinia, herpes symplex virus, fowlpox, vesicular stomatitis virus, canarypox, poliovirus, adeno-associated virus, and lentivirus and sindbus virus. Of course, non-viral vectors, such as liposomes or even naked DNA, are also useful for delivering IACSD-encoding nucleic acids to cells.

Combining the use of nucleic acids to generate immune responses with other types of therapeutic agents or treatments such as chemotherapy or radiation is also contemplated.

2. Expressing Nucleic Acids Encoding IACSD in Cells

In some embodiments, a vector comprising a nucleic acid encoding the IACSD-binding polypeptide (including a fragment, analog or variant) is introduced into a cell, such as a dendritic cell or a macrophage. When expressed in an antigen-presenting cell, IACSD antigens are presented to T cells eliciting an immune response against IACSD. Such methods are known in the art. For an example of the use of similar methods with tumor-specific antigens, see U.S. Pat. No. 6,300,090. The vector encoding IACSD may be introduced into the antigen presenting cells in vivo. Alternatively, antigen-presenting cells may be loaded with IACSD-binding polypeptides or a nucleic acid encoding IACSD-binding polypeptides ex vivo and then introduced into a patient to elicit an immune response against IACSD. Alternatively, the cells presenting IACSD antigen are used to stimulate the expansion of anti-IACSD cytotoxic T lymphocytes (CTL) ex vivo followed by introduction of the stimulated CTL into a patient. Examples of this alternative method using tumor-specific antigens are demonstrated in U.S. Pat. No. 6,306,388. As above, combining this type of therapy with other types of therapeutic agents or treatments such as chemotherapy or radiation is also contemplated.

### Diseases Amenable to Anti-IACSD Immunotargeting

In one aspect, the present invention provides reagents and methods useful for treating diseases and conditions wherein a subset of B-cells associated with the disease or disorder express IACSD. These diseases can include cancers, and other hyperproliferative conditions, such as hyperplasia, psoriasis, contact dermatitis, immunological disorders, and infertility. Whether the subset of B-cells associated with a disease or condition express IACSDs can be determined using the diagnostic methods described herein.

Quantification of IACSD-encoding mRNA and protein expression levels in diseased cells, tissue or fluid (blood, lymphatic fluid, etc.) can be used to determine if the patient will be responsive to IACSD immunotherapy. Methods for detecting and quantifying the expression of IACSD-encoding mRNA or protein use standard nucleic acid and protein detection and quantitation techniques that are well-known in the art and are described in Sambrook et al., Molecular Cloning: A Laboratory Manual, Cold Spring Harbor Laboratory, NY (1989) or Ausubel et al., Current Protocols in Molecular Biology, John Wiley & Sons, New York, N.Y. (1989), both of which are incorporated herein by reference in their entirety. Standard methods for the detection and quantification of mRNA include in situ hybridization using labeled IACSD riboprobes, Northern blot and related techniques using IACSD polynucleotide probes, RT-PCR analysis using IACSD-specific primers, and other amplification detection methods, such as branched chain DNA solution hybridization assays, transcription-mediated amplification, microarray products, such as oligos, cDNAs, and monoclonal antibodies, and real-time PCR. Standard methods for the detection and quantification of IACSD protein include western blot analysis, immunocytochemistry, and a variety of immunoassays, including enzyme-linked immunosorbant assay (ELISA), radioimmuno assay (RIA), and specific enzyme immunoassay (EIA). Peripheral blood cells can also be analyzed for IACSD expression using flow cytometry using, for example, immunomagnetic beads specific for IACSD or biotinylated IACSD antibodies.

In one embodiment, the disease or disorder is a B-cell dependent cancer. Cancer, a leading cause of death in the United States, causes over a half-million deaths annually. As the population ages, the numbers of deaths due to cancer are expected to rise significantly. Cancer is a general term and encompasses various types of malignant neoplasms, most of which invade surrounding tissues, may metastasize to several sites, and are likely to recur after attempted removal and to cause death of the patient unless adequately treated. Cancer can develop in any tissue of any organ at any age. Once a cancer diagnosis is made, treatment decisions are paramount and successful therapy focuses on the primary tumor and its metastases. Various types of cancer treatments have been developed to improve the survival and quality of life of cancer patients. Advances in cancer treatment include new cytotoxic agents and new surgical and radiotherapy techniques. However, many of these treatments have substantial emotional and physical drawbacks and treatment failure remains a common occurrence. Such shortcomings have driven cancer researchers and caregivers to develop new and effective ways of treating cancer.

The cancers treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Tumors or neoplasms include growths of tissue cells in which the multiplication of the cells is uncontrolled and progressive. Some such growths are benign, but others are termed “malignant” and may lead to death of the organism. Malignant neoplasms or “cancers” are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and greater loss of their organization relative to one another and their surrounding tissues. This property is generally called “anaplasia.”

The invention is particularly illustrated herein in reference to treatment of certain types of experimentally defined cancers. In these illustrative treatments, standard state-of-the-art in vitro and in vivo models have been used. These methods can be used to identify agents that can be expected to be efficacious in in vivo treatment regimens. However, it will be understood that the method of the invention is not limited to the treatment of these tumor types, but extends to any B-cell derived cancer. As demonstrated in the Examples, IACSDs are expressed in a subset of primary B-cells and B-cell related disorders. Leukemias can result from uncontrolled B-cell proliferation initially within the bone marrow before disseminating to the peripheral blood, spleen, lymph nodes and finally to other tissues. Uncontrolled B-cell proliferation also may result in the development of lymphomas that arise within the lymph nodes and then spread to the blood and bone marrow. Immunotargeting IACSDs on the subset of B-cells is useful in treating B-cell malignancies, leukemias, lymphomas and myelomas including, but not limited to, the following malignancies listed by the World Health Organization (WHO): Precusor B lymphoblastic leukemia/lymphoma, chronic lymphocytic leukemis/lymphoma, prolymphocytic leukemia, lymphoplasmacytic lymphoma/leukemia, marginal zone lymphoma, hairy cell leukemia, multiple myeloma, /plasmacytoma, malt type lymphoma, monocytoid nodal marginal zone lymphoma, follicular lymphomas, mantle cell lymphoma, diffuse large cell lymphomas, and Burkitt's lymphomas and leukemias. Other B-cell-related malignancies that may be treated in accordance with the present invention include, cutaneous B-cell lymphoma, primary follicular cutaneous B-cell lymphoma, B lineage acute lymphoblastic leukemia (ALL), B-cell non-Hodgkin's lymphoma (NHL), acute lymphoblastic leukemia, primary thyroid lymphoma, intravascular malignant lymphomatosis, splenic lymphoma, Hodgkin's Disease, and intragraft angiotropic large-cell lymphoma.

Autoimmune diseases can be associated with hyperactive B-cell activity that results in autoantibody production and cell-mediated immunity. Inhibition of the development of autoantibody-producing cells or proliferation of such cells may be therapeutically effective in decreasing the levels of autoantibodies in autoimmune diseases including, but not limited to, systemic lupus erythematosis, rheumatoid arthritis, scleroderma, polyarteritis, amyloidosis, Sjogrens syndrome, mixed connective tissue diseases, immune hemolytic anemia, immune thrombocytopenia, immune coagulopathies, immune cytopenias, polymyositis, dermatositis, immune infertility, diabetes mellitus, glomerulonephritis, myasthenia gravis, multiple sclerosis, immune demyelinating diseases, chronic active hepatitis, immune inflammatory bowel diseases, Chron's disease, ulcerative colotis, drug induce autoimmune disease's, necrotizing vascular diseases, erythema multiforme, bukllous skin diseases, eczema, atopic dermatitis, urticaria, angioedema, erythema nodosum, atherosclerosis related diseases, transplant rejection, drug reactions, transfusion reactions, graft-verses-host disease, Graves' disease, asthma, cryoglubulinemia, primary biliary sclerosis, pernicious anemia, Waldenstrom macroglobulinemia, hyperviscosity syndrome, macroglobulinemia, cold agglutinin disease, monoclonal gammopathy of undetermined origin, anetoderma and POEMS syndrome (polyneuropathy, organomegaly, endocrinopathy, M component, skin changes), connective tissue disease, cystic fibrosis, autoimmune pulmonary inflammation, psoriasis, Guillain-Barre syndrome, autoimmune thyroiditis, autoimmune inflammatory eye disease, Goodpasture's disease, Rasmussen's encephalitis, dermatitis herpetiformis, thyoma, autoimmune polyglandular syndrome type 1, primary and secondary membranous nephropathy, cancer-associated retinopathy, autoimmune hepatitis type 1, mixed cryoglobulinemia with renal involvement, cystoid macular edema, endometriosis, IgM polyneuropathy (including Hyper IgM syndrome), demyelinating diseases, angiomatosis, and monoclonal gammopathy.

Where a B cell disorder is related to IgM, the anti-IACSD antibody in accordance with the present invention may recognize a peptide from IgM on the cell surface of a B cell, but not recognize secreted IgM or IgM that is shed into the blood of the individual. Accordingly, the antibody targets a specific subset of B cells associated with the disorder. While not wishing to be limited, the following diseases have been associated with IgM: B-cell leukemias and lymphomas; non-Hodgkin's lymphomas including lymphoplasmacytic lymphoma; Waldenstrom's macrogobulenimia; chronic lymphocytic leukemia; prolymphocytic leukemia; marginal zone lymphoma; hairy cell leukemia; MALT lymphoma; monocytoid nodal marginal zone lymphoma; follicular lymphoma; small cell lymphoma; mixed cell lymphoma; large cell lymphoma; plasmacytoma; mantle cell lymphoma; diffuse large cell lymphoma; Burkitts lymphoma and leukemia; cutaneous B-cell lymphoma; B lineage acute lymphoblastic lymphoma; Hodgkin's disease; IgM polyneuropathy (including Hyper IgM syndrome); mixed cryoglobulinemia with renal involvement; graft-verses-host disease; hyperviscosity syndrome; macroglobulinemia; and cold agglutinin disease. Therefore, individuals having or suspected of having these diseases may benefit from targeted treatment using the anti-IACSD antibody specific to a peptide from membrane-bound IgM.

Where a B cell disorder is related to IgG, the anti-IACSD antibody in accordance with the present invention may recognize a peptide from IgG on the cell surface of a B cell, but not recognize secreted IgG or IgG that is shed into the blood of the individual. Accordingly, the antibody targets a specific subset of B cells associated with the disorder. While not wishing to be limited, the following diseases have been associated with IgG: Auto immune diseases such as systemic lupus, erythematosis, rheumatoid arthritis, scleroderma, polyarteritis, amyloidosis, and Sjogren's syndrome; mixed connective diseases; immune hemolytic anemia; immune throbocytopenias; immune coagulopathies; immune cytopenias; polymyositis; dermatitis; immune fertility; diabetes mellitus; glomerulonephritis; myasthenia gravis; multiple sclerosis; immune demyelinating diseases; chronic active hepatitis; immune inflammatory bowel disease; Chrohn's disease; ulcerative colitis; drug induced autoimmune diseases; necrotizing vascular diseases; erythema multiforme; bullous skin diseases; eczema; atopic dermatitis; urticaria; angioedema; erythema nodosum; atherosclerosis related diseases; transplant rejection; drug reactions; transfusion reactions; graft-verses-host disease; Graves' disease; asthma; cryoglubulinemia; primary biliary sclerosis; pernicious anemia; hyperviscosity syndrome; cold agglutinin disease; monoclonal gammopathy of undetermined origin; POEMS syndrome (polyneuropathy, organomegaly, endocrinopathy, M component, skin changes); connective tissue disease; cystic fibrosis; autoimmune pulmonary inflammation, psoriasis; Guillain-Barre syndrome; autoimmune thyroiditis; autoimmune inflammatory eye disease; Goodpasture's disease; Rasmussen's encephalitis; dermatitis herpetiformis; thymoma; autoimmune polyglandular syndrome type 1; primary and secondary membranous nephropathy; cancer-associated retinopathy; autoimmune hepatitis type 1; mixed cryoglobulinemia with renal involvement; cystoid macular edema; endometriosis; demyelinating diseases; angiomatosis; and monoclonal gammopathy. Therefore, individuals having or suspected of having these diseases may benefit from targeted treatment using the anti-IACSD antibody specific to a peptide from membrane-bound IgG.

Where a B cell disorder is related to IgE, the anti-IACSD antibody in accordance with the present invention may recognize a peptide from IgE on the cell surface of a B cell, but not recognize secreted IgE or IgE that is shed into the blood of the individual. Accordingly, the antibody targets a specific subset of B cells associated with the disorder. While not wishing to be limited, the following diseases have been associated with IgE: acute allergic reaction; anaphylactic reactions; bullous skin diseases; eczema; atopic dermatitis; urticaria, angioedema; erythema nodosum; diabetes mellitus; drug reactions; psoriasis; asthma; hayfever allergic rhinitis; and monoclonal gammopathy. Therefore, individuals having or suspected of having these diseases may benefit from targeted treatment using the anti-IACSD antibody specific to a peptide from membrane-bound IgE.

Where a B cell disorder is related to IgA, the anti-IACSD antibody in accordance with the present invention may recognize a peptide from IgA on the cell surface of a B cell, but not recognize secreted IgA or IgA that is shed into the blood of the individual. Accordingly, the antibody targets a specific subset of B cells associated with the disorder. While not wishing to be limited, the following diseases have been associated with IgA: amyloidosis; glomerulonephritis; chronic active hepatitis; immune inflammatory bowel disease; Chrohn's disease; ulcerative colitis; drug induced autoimmune diseases; erythema multiforme; bullous skin diseases; eczema; atopic dermatitis; urticaria; angioedema; erythema nodosum; autoimmune pulmonary inflammation; psoriasis; primary and secondary membranous nephropathy; hyperviscosity syndrome; and monoclonal gammopathy. Therefore, individuals having or suspected of having these diseases may benefit from targeted treatment using the anti-IACSD antibody specific to a peptide from membrane-bound IgA.

### Administration

The anti-IACSD antibodies used in the practice of a method of the invention may be formulated into pharmaceutical compositions comprising a carrier suitable for the desired delivery method. Suitable carriers include any material which when combined with the anti-IACSD antibodies retains the function of the antibody and is non-reactive with the subject's immune systems. Examples include, but are not limited to, any of a number of standard pharmaceutical carriers such as sterile phosphate buffered saline solutions, bacteriostatic water, and the like.

The anti-IACSD antibody formulations may be administered via any route capable of delivering antibodies to the diseased site. Potentially effective routes of administration include, but are not limited to, intravenous, intraperitoneal, intramuscular, intratumor, intradermal, and the like. The preferred route of administration is by intravenous injection. A preferred formulation for intravenous injection comprises anti-IACSD antibodies in a solution of preserved bacteriostatic water, sterile unpreserved water, and/or diluted in polyvinylchloride or polyethylene bags containing 0.9% sterile sodium chloride for Injection, USP. The anti-IACSD antibody preparation may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the anti-IACSD antibody preparation via an acceptable route of administration such as intravenous injection (IV), typically at a dose in the range of about 0.1 to about 10 mg/kg body weight; however, other exemplary doses in the range of 0.01 mg/kg to about 100 mg/kg are also contemplated. Doses in the range of 10-500 mg mAb per week may be effective and well tolerated. As a non-limiting example, Rituximab (Rituxan™), a chimeric CD20 antibody used to treat B-cell lymphoma, non-Hodgkin's lymphoma, and relapsed indolent lymphoma, is typically administered at 375 mg/m² by IV infusion once a week for 4 to 8 doses. Sometimes multiple courses are desirable or necessary. Thus, an effective dosage range for Rituxan™ would be 50 to 500 mg/m². Similar dosage ranges are expected for the antibody preparations of the present invention. Another example of a dosage regime that may be used is that used with Trastuzumab. Based on clinical experience with Trastuzumab (Herceptin™), a humanized monoclonal antibody used to treat HER2 (human epidermal growth factor 2)-positive metastatic breast cancer with an initial loading dose of approximately 4 mg/kg patient body weight IV followed by weekly doses of about 2 mg/kg IV is adequate. Similarly, this dosage regime may be used with anti-IACSD mAb preparations of the present invention. Preferably, the initial loading dose is administered as a 90-minute or longer infusion. The periodic maintenance dose may be administered as a 30-minute or longer infusion, provided the initial dose was well tolerated. However, as is known in the art, various factors will influence the ideal dose regimen in a particular case. Such factors may include, for example, the binding affinity and half-life of the antibodies used, the degree of IACSD expression in the patient, the desired steady-state antibody concentration level, frequency of treatment, and the influence of chemotherapeutic agents used in combination with the treatment method of the invention.

Treatment can also involve anti-IACSD antibodies conjugated to radioisotopes. As a non-limiting example, studies using radiolabeled-anticarcinoembryonic antigen (anti-CEA) monoclonal antibodies provide a dosage guideline for tumor regression of 2-3 infusions of 30-80 mCi/m² (Behr et al. Clin, Cancer Res. 5(10 Suppl.): 3232s-3242s (1999); Juweid et al., J. Nucl. Med. 39:34-42 (1998)).

Alternatively, dendritic cells transfected with mRNA encoding IACSD can be used as a vaccine to stimulate T-cell mediated responses. For example, studies with dendritic cells transfected with prostate-specific antigen mRNA suggest 3 cycles of intravenous administration of 1×10⁷⁻⁵×10⁷ cells for 2-6 weeks concomitant with an intradermal injection of 10⁷ cells may provide a suitable dosage regimen (Heiser et al., J. Clin. Invest. 109:409-417 (2002); Hadzantonis and O'Neill, Cancer Biother. Radiopharm. 1:11-22 (1999)). Other exemplary doses of between 1×10⁵ to 1×10⁹ or 1×10⁶ to 1×10⁸ cells are also contemplated.

Naked DNA vaccines using plasmids encoding IACSD can induce an immunologic response. Administration of naked DNA by direct injection into the skin and muscle is not associated with limitations encountered using viral vectors, such as the development of adverse immune reactions and risk of insertional mutagenesis (Hengge et al., J. Invest. Dermatol. 116:979 (2001)). Studies have shown that direct injection of exogenous cDNA into muscle tissue results in a strong immune response and protective immunity. Physical (gene gun, electroporation) and chemical (cationic lipid or polymer) approaches have also been developed to enhance efficiency and target cell specificity of gene transfer by plasmid DNA. Plasmid DNA can further be administered to the lungs by aerosol delivery. Gene therapy by direct injection of naked or lipid-coated plasmid DNA is envisioned for the prevention, treatment, and cure of diseases such as cancer, acquired immunodeficiency syndrome, cystic fibrosis, cerebrovascular disease, and hypertension. As a non-limiting example, HIV-1 DNA vaccine dose-escalating studies indicate administration of 30-300 μg/dose as a suitable therapy (Weber et al., Eur. J. Clin. Microbiol. Infect. Dis. 20: 800). Furthermore, naked DNA injected intracerebrally into the mouse brain provides expression of a reporter protein, where the expression is dose-dependent and maximal for 150 μg of injected DNA (Schwartz et al., Gene Ther. 3:405-411 (1996)). Nevertheless, DNA does not need to be in its naked form. DNA may be in a plasmid. For example, gene expression in mice after intramuscular injection of nanospheres containing 1 microgram of beta-galactosidase plasmid was greater and more prolonged than was observed after an injection with an equal amount of naked DNA or DNA complexed with Lipofectamine (Truong et al., Hum. Gene Ther. 9:1709-1717 (1998)). In a study of plasmid-mediated gene transfer into skeletal muscle as a means of providing a therapeutic source of insulin, four plasmid constructs comprising a mouse furin cDNA transgene and rat proinsulin cDNA were injected into the calf muscles of male Balb/c mice, where an optimal dose for most constructs was 100 micrograms plasmid DNA (Kon et al. J. Gene Med. 1: 186-194 (1999)). The doses set forth above may be used with either naked or plasmid DNA. Moreover, exemplary doses of 1-1000 μg/dose or 10-500 μg/dose are also contemplated.

1. IACSD Targeting Compositions

Compositions for targeting IACSD-expressing B-cells are within the scope of the present invention. For example, such compositions may comprise a therapeutically or prophylactically effective amount an antibody, or a fragment, variant, derivative or fusion thereof as described herein, in admixture with a pharmaceutically acceptable agent. Typically, the IACSD targeting element will be sufficiently purified for administration to an animal.

A pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetraacetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapol); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. All of these formulation materials are generally well known in the art.

An optimal pharmaceutical composition will be determined by one skilled in the art depending upon, for example, the intended route of administration, delivery format, and desired dosage. See, for example, Remington's Pharmaceutical Sciences, 18th Edition, Ed. A. R. Gennaro, Mack Publishing Company, (1990). Such compositions may influence the physical state, stability, rate of in vivo release, and rate of in vivo clearance of the IACSD targeting element.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, IACSD targeting element compositions may be prepared for storage by mixing the selected composition having the desired degree of purity with optional formulation agents in the form of a lyophilized cake or an aqueous solution. Further, the binding agent product may be formulated as a lyophilizate using appropriate excipients such as sucrose.

The pharmaceutical compositions can be selected for parenteral delivery. Alternatively, the compositions may be selected for inhalation or for delivery through the digestive tract, such as orally. The preparation of such pharmaceutically acceptable compositions is within the skill of the art. Generally, the formulation components are present in concentrations that are acceptable to the site of administration. For example, buffers are used to maintain the composition at physiological pH or at slightly lower pH, typically within a pH range of from about 5 to about 8. When parenteral administration is contemplated, the therapeutic compositions for use in this invention may be in the form of a pyrogen-free, parenterally acceptable aqueous solution comprising the IACSD targeting element in a pharmaceutically acceptable vehicle. A particularly suitable vehicle for parenteral injection is sterile distilled water in which an IACSD targeting element is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, which provides for the controlled or sustained release of the product, which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

In yet another embodiment, a pharmaceutical composition may be formulated for inhalation. For example, an IACSD targeting element may be formulated as a dry powder for inhalation. Polypeptide or nucleic acid molecule inhalation solutions may also be formulated with a propellant for aerosol delivery. In another embodiment, solutions may be nebulized. Pulmonary administration is further described in PCT Application No. PCT/JS94/001875, which describes pulmonary delivery of chemically modified proteins.

It is also contemplated that certain formulations may be administered orally. In one embodiment of the present invention, IACSD targeting elements that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the binding agent molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., the dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the IACSD targeting element may be dissolved or suspended in suitable liquids, such as fatty oils, liquid, or liquid polyethylene glycol with or without stabilizers.

Another pharmaceutical composition may involve an effective quantity of IACSD targeting element in a mixture with non-toxic excipients suitable for the manufacture of tablets. By dissolving the tablets in sterile water, or other appropriate vehicle, solutions can be prepared in unit dose form. Suitable excipients for these pharmaceutical compositions include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving IACSD targeting elements in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT/US93/00829, which describes controlled release of porous polymeric microparticles in the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D (−)-3-hydroxybutyric acid. Sustained-release compositions also include liposomes, which can be prepared by any of several methods known in the art. See e.g., Epstein et al., Proc. Natl. Acad. Sci. (USA), 82:3688-3692 (1985); EP 36,676; EP 88,046; EP 143,949 for in depth references covering liposomes.

Generally, the pharmaceutical composition to be used for in vivo administration should be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once a pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried IACSD targeting element and a second container having an aqueous formulation. Also included within the scope of this invention are kits containing single and multi-chambered pre-filled syringes (e.g., liquid syringes).

2. Dosage

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which IACSD targeting element is being used, the route of administration, and the size (body weight, body surface or organ size) and condition (the age and general health) of the patient. Accordingly, clinicians may titer the dosage and modify the route of administration to obtain the optimal therapeutic effect. A typical dosage may range from about 0.1 mg/kg to up to about 100 mg/kg or more, depending on the factors mentioned above. In specific embodiments, the dosage may range from 0.1 mg/kg up to about 100 mg/kg; or 0.01 mg/kg to 1 g/kg; or 1 mg/kg up to about 100 mg/kg or 5 mg/kg up to about 100 mg/kg. In other embodiments, the dosage may range from 10 mCi to 100 mCi per dose for radioimmunotherapy, from about 1×10⁷⁻⁵×10⁷ cells or 1×10⁵ to 1×10⁹ cells or 1×10⁶ to 1×10⁸ cells per injection or infusion, or from 30 μg to 300 μg naked DNA per dose or 1-1000 μg/dose or 10-500 μg/dose, depending on the factors listed above.

For any compound, the therapeutically effective dose can be estimated initially either in cell culture assays or in animal models such as mice, rats, rabbits, dogs, or pigs. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the IACSD targeting element in the formulation used. Typically, a composition is administered until a dosage is reached that achieves the desired effect. The composition may therefore be administered as a single dose or as multiple doses (at the same or different concentrations/dosages) over time, or as a continuous infusion. Further refinement of the appropriate dosage is routinely made. Appropriate dosages may be ascertained through use of appropriate dose-response data.

3. Routes of Administration

The route of administration of the pharmaceutical composition is in accord with known methods, e.g. orally, through injection by intravenous, intraperitoneal, intracerebral (intra-parenchymal), intracerebroventricular, intramuscular, intra-ocular, intra-arterial, intraportal, intralesional routes, intramedullary, intrathecal, intraventricular, transdermal, intraplural, subcutaneous, intranasal, enteral, topical, sublingual, urethral, vaginal, or rectal means, by sustained release systems or by implantation devices or generally by injection into any compartment with effusions, which could include any fluid anywhere in the body. Where desired, the compositions may be administered by bolus injection or continuously by infusion, or by implantation device.

Alternatively or additionally, the composition may be administered locally via implantation of a membrane, sponge, or another appropriate material on to which the IACSD targeting element has been absorbed or encapsulated. Where an implantation device is used, the device may be implanted into any suitable tissue or organ, and delivery of the IACSD targeting element may be via diffusion, timed-release bolus, or continuous administration.

In some cases, it may be desirable to use pharmaceutical compositions in an ex vivo manner. In such instances, cells, tissues, or organs that have been removed from the patient are exposed to the pharmaceutical compositions after which the cells, tissues and/or organs are subsequently implanted back into the patient.

In other cases, an IACSD targeting element can be delivered by implanting certain cells that have been genetically engineered to express and secrete the polypeptide. Such cells may be animal or human cells, and may be antilogous, heterologous, or xenogeny. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

### Combination Therapy

IACSD targeting agents of the invention can be utilized in combination with other therapeutic agents. These other therapeutics include, for example radiation treatment, chemotherapeutic agents, as well as other growth factors.

In one embodiment, anti-IACSD antibody is used as a radiosensitizer. In such embodiments, the anti-IACSD antibody is conjugated to a radiosensitizing agent. The term “radiosensitizer,” as used herein, is defined as a molecule, preferably a low molecular weight molecule, administered in therapeutically effective amounts to increase the sensitivity of the cells to be radiosensitized to electromagnetic radiation and/or to promote the treatment of diseases that are treatable with electromagnetic radiation. Diseases that are treatable with electromagnetic radiation include neoplastic diseases, benign and malignant tumors, and cancerous cells.

The terms “electromagnetic radiation” and “radiation” as used herein include, but are not limited to, radiation having the wavelength of 10⁻²⁰ to 100 meters. Preferred embodiments of the present invention employ the electromagnetic radiation of gamma-radiation (10⁻²⁰ to 10⁻¹³ m), X-ray radiation (10⁻¹² to 10⁻⁹ m), ultraviolet light (10 nm to 400 μm), visible light (400 nm to 700 μm), infrared radiation (700 nm to 1.0 mm), and microwave radiation (1 mm to 30 cm).

Radiosensitizers are known to increase the sensitivity of cancerous cells to the toxic effects of electromagnetic radiation. Many cancer treatment protocols currently employ radiosensitizers activated by the electromagnetic radiation of X-rays. Examples of X-ray activated radiosensitizers include, but are not limited to, the following: metronidazole, misonidazole, desmethylmisonidazole, pimonidazole, etanidazole, nimorazole, mitomycin C, RSU 1069, SR 4233, E009, RB 6145, nicotinamide, 5-bromodeoxyuridine (BUdR), 5-iododeoxyuridine (IUdR), bromodeoxycytidine, fluorodeoxyuridine (FUdR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin(r), benzoporphyrin derivatives, NPe6, tin etioporphyrin (SnET2), pheophorbide-a, bacteriochlorophyll-a, naphthalocyanines, phthalocyanines, zinc phthalocyanine, and therapeutically effective analogs and derivatives of the same.

Chemotherapy treatment can employ anti-neoplastic agents including, for example, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as triethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophyllotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone, and anthracycline.

Combination therapy with growth factors can include combination with cytokines, lymphokines, growth factors, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF0, TNF1, TNF2, G-CSF, Meg-CSF, GM-CSF, thrombopoietin, stem cell factor, and erythropoietin. Other compositions can include known angiopoietins, for example, vascular endothelial growth factor (VEGF). Growth factors include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2, cytokine-induced neutrophil chemotactic factor 2, endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor 1, glial cell line-derived neutrophic factor receptor 2, growth related protein, growth related protein, growth related protein, growth related protein, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor, nerve growth factor nerve growth factor receptor, neurotrophin-3; neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor, platelet derived growth factor receptor, pre-B cell growth stimulating factor, stem cell factor, stem cell factor receptor, transforming growth factor, transforming growth factor, transforming growth factor 1, transforming growth factor 1.2, transforming growth factor. 2, transforming growth factor 3, transforming growth factor 5, latent transforming growth factor 1, transforming growth factor binding protein I, transforming growth factor binding protein II, transforming growth factor binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

The present invention contemplates the administration of IACSD targeting agents separately, sequentially, or simultaneously with, radiation, chemotherapeutic agents, or growth factors. Likewise, the radiation, chemotherapeutic agent, or growth factor may be administered with the IACSD targeting agent in any order or concurrently, or as conjugates, as described above.

### Diagnostic Uses of IACSDs

1. Assays for Determining IACSD-Expression Status

Determining the status of IACSDs expression patterns in an individual may be used to diagnose cancer and may provide prognostic information useful in defining appropriate therapeutic options. Similarly, the expression status of IACSDs may provide information useful for predicting susceptibility to particular disease stages, progression, and/or tumor aggressiveness. The invention provides methods and assays for determining IACSDs expression status and diagnosing cancers that express IACSDs.

In one aspect, the invention provides assays useful in determining the presence of cancer in an individual, comprising detecting IACSD-encoding mRNA or protein expression in a test cell or tissue or fluid sample. In one embodiment, the presence of IACSD-encoding mRNA is evaluated in tissue samples of a lymphoma. The presence of significant IACSD expression may be useful to indicate whether the lymphoma is susceptible to IACSD immunotargeting. In a related embodiment, IACSD expression status may be determined at the protein level rather than at the nucleic acid level. For example, such a method or assay would comprise determining the level of IACSD expressed by cells in a test tissue sample and comparing the level so determined to the level of IACSD expressed in a corresponding normal sample. In one embodiment, the presence of IACSD is evaluated, for example, using immunohistochemical methods. Anti-IACSD antibodies capable of detecting IACSD expression may be used in a variety of assay formats well known in the art for this purpose.

Peripheral blood containing the subset of B-cells may be conveniently assayed for the presence of cancer cells, including lymphomas and leukemias, using RT-PCR to detect IACSD expression. The presence of RT-PCR amplifiable IACSD-encoding mRNA provides an indication of the presence of one of these types of cancer. A sensitive assay for detecting and characterizing carcinoma cells in blood may be used, such as that demonstrated in Racila et al., Proc. Natl. Acad. Sci. USA 95: 4589-4594 (1998). This assay combines immunomagnetic enrichment with multiparameter flow cytometric and immunohistochemical analyses, and is highly sensitive for the detection of cancer cells in blood, reportedly capable of detecting one epithelial cell in 1 ml of peripheral blood.

A related aspect of the invention is directed to predicting susceptibility to developing cancer in an individual. In one embodiment, a method for predicting susceptibility to cancer comprises detecting IACSD-encoding mRNA or IACSD in a tissue sample, its presence indicating susceptibility to cancer, wherein the degree of IACSD-encoding mRNA expression present is proportional to the degree of susceptibility.

Yet another related aspect of the invention is directed to methods for assessment of tumor aggressiveness. In one embodiment, a method for gauging aggressiveness of a tumor comprises determining the level of IACSD-encoding mRNA or IACSD protein expressed by the subset of B-cells in a sample of the tumor, comparing the level so determined to the level of IACSD-encoding mRNA or IACSD protein expressed in a corresponding normal tissue taken from the same individual or a normal tissue reference sample, wherein the relative degree of IACSD-encoding mRNA or IACSD protein expression in the tumor sample indicates the degree of aggressiveness.

Methods for detecting and quantifying the expression of IACSD-encoding mRNA or protein are described herein and use standard nucleic acid and protein detection and quantification technologies well known in the art. Standard methods for the detection and quantification of IACSD-encoding mRNA include in situ hybridization using labeled IACSD-encoding riboprobes, Northern blot and related techniques using IACSD-encoding polynucleotide probes, RT-PCR analysis using primers specific for IACSD-encoding polynucleotides, and other amplification type detection methods, such as, for example, branched DNA, SISBA, TMA, and microarray products of a variety of sorts, such as oligos, cDNAs, and monoclonal antibodies. In a specific embodiment, real-time RT-PCR may be used to detect and quantify IACSD-encoding mRNA expression. Standard methods for the detection and quantification of protein may be used for this purpose. In a specific embodiment, polyclonal or monoclonal antibodies specifically reactive with the wild type IACSD may be used in an immunohistochemical assay of biopsied tissue.

2. Medical Imaging

Anti-IACSD antibodies and fragments thereof are useful in medical imaging of sites expressing IACSD. Such methods involve chemical attachment of a labeling or imaging agent, such as a radioisotope, which include ⁶⁷Cu, ⁹⁰Y, ²⁵I, ¹³¹I, ¹⁸⁶Re, ¹⁸⁸Re, ²¹¹At, and ²¹²Bi, administration of the labeled antibody and fragment to a subject in a pharmaceutically acceptable carrier, and imaging the labeled antibody and fragment in vivo at the target site. Radiolabelled anti-IACSD antibodies or fragments thereof may be particularly useful in in vivo imaging of IACSD expressing cancers, such as lymphomas or leukemias. Such antibodies may provide highly sensitive methods for detecting metastasis of IACSD-expressing cancers either by external imaging or biopsy or detection of localized radioactivity.

Upon consideration of the present disclosure, one of skill in the art will appreciate that many other embodiments and variations may be made in the scope of the present invention. Accordingly, it is intended that the broader aspects of the present invention not be limited to the disclosure of the following examples.

### EXAMPLES

### Example 1

### Production of IACSD-Specific Antibody for IgM-Related B Cell Disorders

In this example, monoclonal antibodies specific to a IACSD corresponding to membrane-bound IgM were generated. Six immunogens were used: (1) ATCC cell line, CRL-2261 (B-cell lymphocytic lymphoma/leukemia cells); (2) NP-40 lysate of CRL cells; (3) Membrane IgM immunoaffinity enriched lysate of CRL cells; (4) KLH-peptide; (5) GST-peptide; (6) MAP-peptide. Peptide EGEVSADEEGFEN (SEQ ID NO: 7) (referred to in this example as the “peptide”) was conjugated to KLH-, GST-, and MAP. The specificity of this sequence for membrane IgM was confirmed by searching the peptide against all human proteins in GenBank.

One hundred and two fusions of mouse splenocytes/sp20 cells yielded 28 clones producing monoclonal antibodies reactive with the peptide. Fusions using immunogens 1-4 did not result in generation of monoclonal antibodies specific for the target peptide. However, fusions using GST-peptide as the immunogen produced 15 positive hybridoma clones (Table 2) and fusions using MAP-peptide as the immunogen produced 13 clones (Table 3). These 28 clones were proven to produce monoclonal antibodies specific for the target peptide.

TABLE 2
Monoclonal Antibodies Obtained from
GST-Derived Peptide Immunogens

| | | CRL-IgM | KLH-Pep | Human Serum |
|---|---|---|---|---|
| Clone # | GRI-Desig | Reactivity | Reactivity | Reactivity |
| 88-55 | GRI-SW-M-1 | + | + | − |
| 87-35 | GRI-SW-M-2 | + | + | − |
| 88-82 | GRI-SW-M-3 | + | + | − |
| 89-11 | GRI-SW-M-4 | + | + | − |
| 89-15 | GRI-SW-M-5 | + | + | − |
| 89-52 | GRI-SW-M-6 | + | + | − |
| 89-60 | GRI-SW-M-7 | + | + | − |
| 89-66 | GRI-SW-M-8 | + | + | − |
| 89-71* | GRI-SW-M-28 | + | + | ND |
| 90-24 | GRI-SW-M-9 | + | + | − |
| 95-20 | GRI-SW-M-10 | + | + | − |
| 90-39 | GRI-SW-M-11 | + | + | − |
| 90-49 | GRI-SW-M-12 | + | + | − |
| 95-27 | GRI-SW-M-13 | + | + | − |
| 96-49 | GRI-SW-M-14 | + | + | − |

*Shows growth inhibitory properties.

TABLE 3
Monoclonal Antibodies Obtained from
MAP-Derived Peptide Immunogens

| | | CRL-IgM | KLH-Pep | Human Serum |
|---|---|---|---|---|
| Clone # | GRI-Desig | Reactivity | Reactivity | Reactivity |
| 97-147 | GRI-SW-M-15 | + | + | ND |
| 97-31 | GRI-SW-M-16 | + | + | − |
| 97-61 | GRI-SW-M-17 | + | + | − |
| 99-82 | GRI-SW-M-18 | + | + | − |
| 99-94 | GRI-SW-M-19 | + | + | − |
| 100-14 | GRI-SW-M-20 | + | + | − |
| 100-23 | GRI-SW-M-21 | + | + | ND |
| 100-24 | GRI-SW-M-22 | + | + | − |
| 100-25 | GRI-SW-M-23 | + | + | − |
| 100-40 | GRI-SW-M-24 | + | + | ND |
| 100-63 | GRI-SW-M-25 | + | + | − |
| 100-69 | GRI-SW-M-26 | + | + | − |
| 102-19 | GRI-SW-M-27 | + | + | ND |

To verify specificity of the antibody for membrane IgM, five criteria were established: (1) The antibody must react with the peptide on the immunogen (GST-peptide or MAP-peptide); (2) The antibody must bind to the peptide on a KLH-peptide construct; (3) The antibody must bind to the peptide on the native protein by ELISA of membrane IgM derived from CRL-2261 cells; (4) The antibody must not be reactive with human serum proteins, as shown by inhibition assay with KLH-peptide or membrane IgM derived from CRL cells; and (5) The antibody must not be reactive with serum IgM in ELISA. The rationale for this screening scheme was to eliminate monoclonal antibodies binding to human serum proteins including serum IgM and collect all the monoclonal antibodies binding specifically to the IACSD.

The results are of this screening are shown in Tables 2 and 3. First, screening against the GST-peptide or MAP-peptide was conducted as part of the hybridoma selection process. Only those clones which bind to the immunogen are carried forward in the screening. Therefore, all of the 28 clones selected for further screening were positive for either GST-peptide or MAP-peptide binding (data not shown). Second, specific monoclonal antibody reactivity was examined by assaying binding of the antibody to the peptide on a KLH-peptide construct. The 28 clones identified initially showed binding to the peptide on a KLH-peptide construct (Tables 2 and 3, fourth column). Third, specific monoclonal antibody reactivity was examined by assaying binding of the antibody to the native IgM protein. Membrane IgM was derived from CRL-2261 cells and binding was assayed by ELISA, All 28 clones showed binding to the native IgM derived from CRL cells (Tables 2 and 3, third column). Fourth, the antibodies were tested for reactivity with serum proteins by inhibition assays using normal human serum to block antibody binding to KLH-peptide or immunoadsorbed membrane IgM derived from CRL cells. Most clones (23 of 28) showed no reactivity to human sera (Tables 2 and 3, fifth column). Reactivity was not measured for the remaining 7 clones (ND). Finally, monoclonal antibody clones were shown not to be reactive with serum IgM which does not carry the target peptide using immunoadsorbed serum derived IgM in ELISA assays (data not shown).

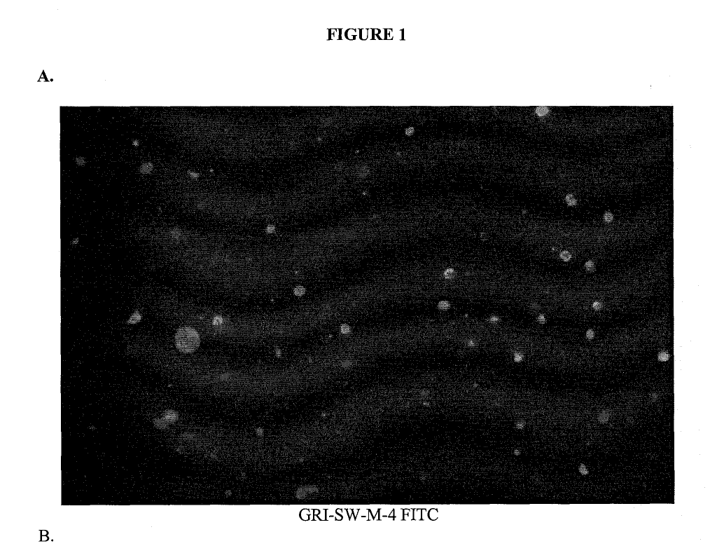

The specificity of the anti-IACSD antibody for cells in vitro was analyzed using fluorescence microscopy. Fluorescein (FITC) conjugated antibodies from clone GRI-SW-M-4 were prepared. The antibodies were added to a preparation of CRL-2261 cells. While membrane IgM reactivity in fluorescent staining experiments are usually described as “dim” in intensity, these antibodies exhibited dim to moderate reactivity in a heterogeneous pattern by fluorescence staining (FIG. 1). Thus, anti-IACSD antibodies have been shown to specifically bind to membrane-associated IgM in intact cells.

### Example 2

### Cell Growth Inhibitory and Cytotoxic Effects of IACSD-Specific Antibodies

A MTT assay was used to measure growth inhibition and cytotoxic effects of anti-IACSD antibodies in CRL-2261 cells. MTT is a calorimetric assay using the dye Yellow MTT (3-(4,5-Dimethylthiazol-2-yl)-2,5-diphenyltetrazolium bromide, a tetrazole), which is reduced to purple formazan in mitochondria. CRL-2261 cells were counted and adjusted to 48,000 cells/well. Each sample was tested in quadruplicate. The following samples were assayed: (1) 10 μl of monoclonal supernatant was added to 8 wells; (2) 10 μl of regular media was added to 4 of the “−Rituxan” wells; (3) 10 μl of a 100 μg/ml Rituxan solution in regular media was added to 4 wells labeled “+Rituxan”; (4) a control series with no supernatant; and (5) control series with SP20 supernatant. The plates were incubated at 37° C., 5% CO₂ for 3 days. Following incubation, 10 μl of MTT solution was added to each well and the plates were put back in the incubator for 2 hours. The reaction was stopped by adding 100 μl of stop solution. The plates were incubated at room temperature, in the dark, for 2 hours and then the absorbance was read at 560 nm minus reading at 650 nm.

The results are shown in Table 4. The average absorbance of all wells containing the same sample are indicated. Samples that were treated with Rituxan typically showed some modest growth inhibition and cytotoxicity. However, clone supernatant 89-71 had significant cytotoxic effect, with and without Rituxan also present.

TABLE 4
Cytotoxic Effects of Monoclonal Supernatants

| Clone Supernatant | −Rituxan | +Rituxan |
|---|---|---|
| 89-66 | 0.40 | 0.34 |
| 89-71 | 0.02 | 0.03 |
| 90-24-2 | 0.47 | 0.40 |
| 89-52-2 | 0.50 | 0.40 |
| 88-55-1 | 0.49 | 0.40 |
| 88-79-1 | 0.47 | 0.34 |
| control | 0.49 | 0.34 |
| control w/sp20 sup. | 0.52 | 0.45 |

The results indicate that antibody-binding alone may be sufficient to cause cytotoxic effects and kill cells directly. While not wishing to be bound by theory, it is believed that the some antibodies may compete for binding with other membrane proteins that are associated with the membrane IgM. If the monoclonal antibody affinity exhibits greater affinity than these other proteins and binds to IgM, then the membrane IgM is internalized. The cells are programmed to differentiate, but malignant cells die. This process may explain why the 89-71 monoclonal is able to kill cells directly. Antibodies that have less affinity may still bind to the target, but not compete as effectively against other proteins. Such antibodies would not be expected to have direct cytotoxic effects.

### Example 3

### Production of IACSD-Specific Antibodies

Humanized monoclonal antibodies will be engineered either by CDR-IgG1 human framework engineering (CDR grafting), or by chimerization, or by using phage display technology to identify peptides that specifically bind to IACSD. Phage peptide libraries (strains M13, fl, or fd) obtained from commercial vendors will be screened for their ability to bind to the IACSD peptide. These libraries may consist of random peptide sequences like New England Biolabs (Beverly Mass.) “PhD” libraries or antibody libraries in which phage express scFv regions on their surface. The screening step will be performed on synthetic peptides, purified membrane Immunoglobulin containing the IACSD or cell lines expressing the IACSD. The methods of screening phage are well known in the art. For reviews see, Phage Display: a laboratory manual, Barbas, et al. Cold Spring Harbor Press, (2001), Azzazy & Highsmith, Clinical Biochemistry 35:425 (2002), Siegel, Transfus Clin Biol 9:15 (2002), Baca, et al., J. Biol. Chem. 272#16 10678 (1997), O'Connell, et al., J. Mol. Biol. 321: 49 (2002).

Phage that are found to bind, will be amplified and tested for their ability to bind IACSD using an ELISA assay. ELISA based methods are well known in the at and are described in ELISA Theory and Practice, Crowther, J., Humana Press 1995, and Current Protocols in Immunology John Wiley and Sons, New York, N.Y. (1994). Individual phage that demonstrate strong binding to the IACSD will be sequenced using the Applied Biosystems (Foster City, Calif.) Big Dye Sequencing kit.

The peptide will then be cloned into a human antibody construct in the region of the molecule referred to as the Complemetarity Determining Region (CDR), Popkov, M., et al., J. Immunol. Meth. 291: 137 (2004), using standard cloning techniques that are known in the art and described in Ausubel et al., Current protocols in molecular Biology, John Wiley and Sons, New York, N.Y. (1998).

### Example 4

### In Vitro Antibody-Dependent Cytotoxicity Assay

The ability of an IACSD-specific antibody to induce antibody-dependent cell-mediated cytoxicity (ADCC) can be determined in vitro. ADCC is performed using the CytoTox 96 Non-Radioactive Cytoxicity Assay (Promega; Madison) as well as effector and target cells. Peripheral blood mononuclear cells (PBMC) or neutrophilic polymorphonuclear leukocytes (PMN) are two examples of effector cells that are used in this assay. PBMC is isolated from healthy human donors by Ficoll-Paque gradient centrifugation, and PMN is purified by centrifugation through a discontinuous percoll gradient (70% and 62%) followed by hypotonic lysis to remove residual erythrocytes. RA1 B-cell lymphoma cells or American Type Culture Collection (ATCC) CRL 2261 lymphoma cells (for example) are used as target cells.

RA1 cells are suspended in RPMI 1640 medium supplemented with 2% fetal bovine serum and plated in 96-well V-bottom microtiter plates at 2×10⁴ cells/well. IACSD-specific antibody is added in triplicate to individual wells at 1 μg/ml, and effector cells are added at various effector:target cell ratios (such as 12.5:1 to 50:1). The plates are incubated for 4 hours at 37° C. The supernatants are harvested, lactate dehydrogenase release is determined, and percent specific lysis is calculated using the manufacture's protocols.

### Example 5

### Toxin-Conjugated IACSD-Specific Antibodies

Antibodies to IACSD are conjugated to toxins and the effect of such conjugates in animal models of cancer is evaluated. Chemotherapeutic agents, such as calichemycin and carboplatin, or toxic peptides, such as ricin toxin, are used in this approach. Antibody-toxin conjugates are used to target cytotoxic agents specifically to cells bearing the antigen. The antibody-toxin binds to these antigen-bearing cells, becomes internalized by receptor-mediated endocytosis, and subsequently destroys the targeted cell. In this case, the antibody-toxin conjugate targets IACSD-expressing cells, such as B-cell lymphomas, and delivers the cytotoxic agent to the tumor resulting in the death of the tumor cells.

One such example of a toxin that may be conjugated to an antibody is carboplatin. The mechanism by which this toxin is conjugated to antibodies is described in Ota et al., Asia-Oceania J. Obstet. Gynaecol. 19: 449-457 (1993). The cytotoxicity of carboplatin-conjugated IACSD-specific antibodies is evaluated in vitro, for example, by incubating IACSD-expressing target cells (such as the RA1 B-cell lymphoma cell line) with various concentrations of conjugated antibody, medium alone, carboplatin alone, or antibody alone. The antibody-toxin conjugate specifically targets and kills cells bearing the IACSD antigen, whereas, cells not bearing the antigen, or cells treated with medium alone, carboplatin alone, or antibody alone, show no cytotoxicity.

The antitumor efficacy of carboplatin-conjugated IACSD-specific antibodies is demonstrated in in vivo murine tumor models. Five to six week old, athymic nude mice are engrafted with tumors subcutaneously or through intravenous injection. Mice are treated with the IACSD-carboplatin conjugate or with a non-specific antibody-carboplatin conjugate. Tumor xenografts in the mice bearing the IACSD antigen are targeted and bound to by the IACSD-carboplatin conjugate. This results in tumor cell killing as evidenced by tumor necrosis, tumor shrinkage, and increased survival of the treated mice.

Other toxins are conjugated to IACSD-specific antibodies using methods known in the art. An example of a toxin-conjugated antibody in human clinical trials is CMA-676, an antibody to the CD33 antigen in AML, which is conjugated with calicheamicin toxin.

### Example 6

### Radio-Immunotherapy Using IACSD-Specific Antibodies

Animal models are used to assess the effect of antibodies specific to IACSDs as vectors in the delivery of radionuclides in radio-immunotherapy to treat lymphoma, hematological malignancies, and solid tumors. Human tumors are propagated in 5-6 week old athymic nude mice by injecting a cancerous B-cell line or tumor cells subcutaneously. Tumor-bearing animals are injected intravenously with radiolabeled anti-IACSD antibody (labeled with 30-40 μCi of ¹³¹I, for example). Tumor size is measured before injection and on a regular basis (i.e. weekly) after injection and compared to tumors in mice that have not received treatment. Anti-tumor efficacy is calculated by correlating the calculated mean tumor doses and the extent of induced growth retardation. To check tumor and organ histology, animals are sacrificed by cervical dislocation and autopsied. Organs are fixed in 10% formalin, embedded in paraffin, and thin sectioned. The sections are stained with hematoxylin-eosin.

### Example 7

### Immunotherapy Using IACSD-Specific Antibodies

Animal models are used to evaluate the effect of IACSD-specific antibodies as targets for antibody-based immunotherapy using monoclonal antibodies. Human myeloma cells are injected into the tail vein of 5-6 week old nude mice whose natural killer cells have been eradicated. To evaluate the ability of IACSD-specific antibodies in preventing tumor growth, mice receive an intraperitoneal injection with IACSD-specific antibodies either 1 or 15 days after tumor inoculation followed by either a daily dose of 20 μg or 100 μg once or twice a week, respectively. Levels of human IgG (from the immune reaction caused by the human tumor cells) are measured in the murine sera by ELISA.

The effect of IACSD-specific antibodies on the proliferation of myeloma cells is examined in vitro using a ³H-thymidine incorporation assay. Cells are cultured in 96-well plates at 1×10⁵ cells/ml in 100 μl/well and incubated with various amounts of IACSD antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

The cytotoxicity of the anti-IACSD monoclonal antibody is examined by the effect of complements on myeloma cells using a ⁵¹Cr-release assay. Myeloma cells are labeled with 0.1 mCi ⁵¹Cr-sodium chromate at 37° C. for 1 h. ⁵¹Cr-labeled cells are incubated with various concentrations of anti-IACSD monoclonal antibody or control IgG on ice for 30 min. Unbound antibody is removed by washing with medium. Cells are distributed into 96-well plates and incubated with serial dilutions of baby rabbit complement at 37° C. for 2 h. The supernatants are harvested from each well and the amount of ⁵¹Cr released is measured using a gamma counter. Spontaneous release of ⁵¹Cr is measured by incubating cells with medium alone, whereas maximum ⁵¹Cr release is measured by treating cells with 1% NP-40 to disrupt the plasma membrane. Percent cytotoxicity is measured by dividing the difference of experimental and spontaneous ⁵¹Cr release by the difference of maximum and spontaneous ⁵¹Cr release.

Antibody-dependent cell-mediated cytotoxicity (ADCC) for the anti-IACSD monoclonal antibody is measured using a standard 4 h ⁵¹Cr-release assay. Splenic mononuclear cells from SCID mice are used as effector cells and cultured with or without recombinant interleukin-2 (for example) for 6 days. ⁵¹Cr-labeled target myeloma cells (1×10⁴ cells) are placed in 96-well plates with various concentrations of anti-IACSD monoclonal antibody or control IgG. Effector cells are added to the wells at various effector to target ratios (12.5:1 to 50:1). After 4 h, culture supernatants are removed and counted in a gamma counter. The percentage of cell lysis is determined as above.

### Example 8

### IACSD-Specific Antibodies as Immunosuppressants

Animal models are used to assess the effect of IACSD-specific antibodies block signaling through the IACSD receptor to suppress autoimmune diseases, such as arthritis or other inflammatory conditions, or rejection of organ transplants. Immunosuppression is tested by injecting mice with horse red blood cells (HRBCs) and assaying for the levels of HRBC-specific antibodies. Animals are divided into five groups, three of which are injected with anti-IACSD antibodies for 10 days, and 2 of which receive no treatment. Two of the experimental groups and one control group are injected with either Earle's balanced salt solution (EBSS) containing 5-10×10⁷ HRBCs or EBSS alone. Anti-IACSD antibody treatment is continued for one group while the other groups receive no antibody treatment. After 6 days, all animals are bled by retro-orbital puncture, followed by cervical dislocation and spleen removal. Splenocyte suspensions are prepared and the serum is removed by centrifugation for analysis.

Immunosuppression is measured by the number of B cells producing HRBC-specific antibodies. The Ig isotype (for example, IgM, IgG1, IgG2, etc.) is determined using the IsoDetect™ Isotyping kit (Stratagene, La Jolla, Calif.). Once the Ig isotype is known, murine antibodies against HRBCs are measured using an ELISA procedure. 96-well plates are coated with HRBCs and incubated with the anti-HRBC antibody-containing sera isolated from the animals. The plates are incubated with alkaline phosphatase-labeled secondary antibodies and color development is measured on a microplate reader (SPECTRAmax 250, Molecular Devices) at 405 nm using p-nitrophenyl phosphate as a substrate.

Lymphocyte proliferation is measured in response to the T and B-cell activators concanavalin A and lipopolysaccharide, respectively. Mice are randomly divided into 2 groups, 1 receiving anti-IACSD antibody therapy for 7 days and 1 as a control. At the end of the treatment, the animals are sacrificed by cervical dislocation, the spleens are removed, and splenocyte suspensions are prepared as above. For the ex vivo test, the same number of splenocytes are used, whereas for the in vivo test, the anti-IACSD antibody is added to the medium at the beginning of the experiment. Cell proliferation is also assayed using the ³H-thymidine incorporation assay described above.

### Example 9

### Cytokine Secretion in Response to IACSD Peptide Fragments

Assays are carried out to assess activity of fragments of the IACSD proteins, such as the Ig domain, to stimulate cytokine secretion and to stimulate immune responses in NK cells, B-cells, T cells, and myeloid cells. Such immune responses can be used to stimulate the immune system to recognize and/or mediate tumor cell killing or suppression of growth. Similarly, this immune stimulation can be used to target bacterial or viral infections. Alternatively, fragments of IACSDs that block activation through the IACSDs receptor may be used to block immune stimulation in natural killer (NK), B, T, and myeloid cells.

Fusion proteins containing fragments of IACSDs, such as the Ig domain, are made by inserting a CD33 leader peptide, followed by an IACSD domain fused to the Fc region of human IgG1 into a mammalian expression vector, which is stably transfected into NS-1 cells, for example. The fusion proteins are secreted into the culture supernatant, which is harvested for use in cytokine assays, such as interferon-γ secretion assays.

PBMCs are activated with a suboptimal concentration of soluble CD3 and various concentrations of purified, soluble anti-IACSD monoclonal antibody or control IgG. For IACSD-Ig cytokine assays, anti-human Fc Ig at 5 or 20 μg/ml is bound to 96-well plates and incubated overnight at 4° C. Excess antibody is removed and either IACSD-Ig or control Ig is added at 20-50 μg/ml and incubated for 4 h at room temperature. The plate is washed to remove excess fusion protein before adding cells and anti-CD3 to various concentrations. Supernatants are collected after 48 h of culture and interferon-γ levels are measured by sandwich ELISA, using primary and biotinylated secondary anti-human interferon-γ antibodies as recommended by the manufacturer.

### Example 10

### Diagnostic Methods Using IACSD-Specific Antibodies to Detect IACSD Expression

Expression of IACSDs in tissue samples (normal or diseased) is detected using anti-IACSD antibodies. Samples are prepared for immunohistochemical (IHC) analysis by fixing the tissue in 10% formalin embedding in paraffin, and sectioning using standard techniques. Sections are stained using the IACSD-specific antibody followed by incubation with a secondary horseradish peroxidase (HRP)-conjugated antibody and visualized by the product of the HRP enzymatic reaction.

Expression of IACSD on the surface of cells within a blood sample is detected by flow cytometry. Peripheral blood mononuclear cells (PBMC) are isolated from a blood sample using standard techniques. The cells are washed with ice-cold PBS and incubated on ice with the IACSD-specific polyclonal antibody for 30 min. The cells are gently pelleted, washed with PBS, and incubated with a fluorescent anti-rabbit antibody for 30 min. on ice. After the incubation, the cells are gently pelleted, washed with ice cold PBS, and resuspended in PBS containing 0.1% sodium azide and stored on ice until analysis. Samples are analyzed using a FACScalibur flow cytometer (Becton Dickinson) and CELLQuest software (Becton Dickinson). Instrument settings are determined using FACS-Brite calibration beads (Becton-Dickinson).

Tumors expressing IACSD is imaged using IACSD-specific antibodies conjugated to a radionuclide, such as ¹²³I, and injected into the patient for targeting to the tumor followed by X-ray or magnetic resonance imaging.

### Example 11

### Tumor Imaging Using IACSD-Specific Antibodies

IACSD-specific antibodies are used for imaging IACSD-expressing cells in vivo. Six-week-old athymic nude mice are irradiated with 400 rads from a cesium source. Three days later the irradiated mice are inoculated with 4×10⁷ RA1 cells and 4×10⁶ human fetal lung fibroblast feeder cells subcutaneously in the thigh. When the tumors reach approximately 1 cm in diameter, the mice are injected intravenously with an inoculum containing 100 μCi/10 μg of ¹³¹I-labeled IACSD-specific antibody. At 1, 3, and 5 days postinjection, the mice are anesthetized with a subcutaneous injection of 0.8 mg sodium pentobarbital. The immobilized mice are then imaged in a prone position with a Spectrum 91 camera equipped with a pinhole collimator (Raytheon Medical Systems; Melrose Park, Ill.) set to record 5,000 to 10,000 counts using the Nuclear MAX Plus image analysis software package (MEDX Inc.; Wood Dale, Ill.).

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, particular embodiments encompass not only the entire group listed as a whole, but each member of the group individually and all possible subgroups of the main group. Accordingly, for all purposes, certain embodiments encompass not only the main group, but also the main group absent one or more of the group members. Individual embodiments also envisage the explicit exclusion of one or more of any of the group members.

All references, patents and publications disclosed herein are specifically incorporated by reference thereto. Unless otherwise specified, “a” or “an” means “one or more.”

While preferred embodiments have been illustrated and described, it should be understood that changes and modifications can be made therein in accordance with ordinary skill in the art without departing from the invention in its broader aspects as described herein. The broader aspects of the present invention are defined in the following claims.

What is claimed is:

1. A method comprising administering to an individual an effective amount of a targeting antibody or antibody fragment, wherein the targeting antibody or antibody fragment specifically recognizes an immunoglobulin associated cell-surface determinant on a B cell that is not shed into the blood of the individual or present in the corresponding secreted immunoglobulin.

2. The method of claim 1, wherein the antibody or antibody fragment is humanized.

3. The method of claim 1, wherein the immunoglobulin associated cell-surface determinant is a peptide associated with an immunoglobulin isotype selected from the group consisting of IgA, IgD, IgE, IgG, and IgM.

4. The method of claim 1, wherein the immunoglobulin associated cell-surface determinant is a peptide comprising any of SEQ ID NO: 1-8 or immunogenic fragments thereof or variants having at least 95% amino acid identity to any of SEQ ID NO: 1-8.

5. The method of claim 1, further comprising administering a therapeutically effective amount of a cytotoxic agent, wherein the cytotoxic agent and the targeting antibody or antibody fragment may be administered in any order or concurrently.

6. The method of claim 5, wherein the cytotoxic agent and the targeting antibody or antibody fragment form a conjugate.

7. The method of claim 5, wherein the cytotoxic agent is a chemotherapeutic agent.

8. The method of claim 5, wherein the cytotoxic agent is a radionuclide.

9. The method of claim 1, wherein the individual has or is suspected of having a B cell related disorder.

10. The method of claim 1, further comprising the step of administering at least one additional targeting agent that targets a determinant on the B-cell.

11. The method of claim 10, wherein the at least one additional targeting agent targets the CD20 epitope on the B-cell.

12. A composition comprising an isolated antibody or antibody fragment and a cytotoxic agent, wherein the isolated antibody or antigen binding fragment associates with an immunoglobulin associated cell surface determinant on a B cell that is not shed into the blood of a host or present in the corresponding secreted immunoglobulin.

13. The composition of claim 12, wherein the immunoglobulin associated cell-surface determinant is a peptide associated with an immunoglobulin isotype selected from the group consisting of IgA, IgD, IgE, IgG, and IgM.

14. The composition of claim 12, wherein the immunoglobulin associated cell-surface determinant is a peptide comprising any one of SEQ ID NO: 1-8 or immunogenic fragments thereof or variants having at least 95% amino acid identity to any one of SEQ ID NOS: 1-8.

15. The composition of claim 12, wherein the cytotoxic agent is a chemotherapeutic agent.

16. The composition of claim 12, wherein the cytotoxic agent is a radionuclide.

17. The composition claim 12, wherein the antibody or antibody fragment is humanized.

18. The composition of claim 12, further comprising at least one additional targeting agent that targets a determinant on the B cell.

19. The composition of claim 18, wherein the at least one additional targeting agent targets the CD20 epitope on the B cell.

20. A method comprising:
(a) detecting or measuring in a sample the expression of an immunoglobulin associated cell surface determinant protein that is not shed into the blood of a host or present in the corresponding secreted immunoglobulin or the expression of an immunoglobulin associated cell surface determinant nucleic acid in a cell, wherein the sample is from an individual having or suspected of having a B cell disorder; and
(b) comparing the expression to a standard, wherein the expression of the immunoglobulin associated cell surface determinant protein or nucleic acid relative to the standard is correlated to a B cell disorder.
